(19) United States
(12) Patent Application Publication
(10) Pub. No.: US 2010/0152188 A1
(43) Pub. Date: Jun. 17, 2010

(54) Novel Heterocyclic Compounds

(76) Inventors: Akella Satya Surya Visweswara Srinivas, Sholinganallur (IN); Kasinathan Mathiyazhagan, Tamil Nadu (IN); Duddu Savaraiah Sharada, Tamil Nadu (IN); Thanasekaran Ponpandian, Tamil Nadu (IN); Kulasekharan Revathy, Tamil Nadu (IN); Gaddam Om Reddy, Tamil Nadu (IN); Mani Kamarai, Tamil Nadu (IN); Sriram Raiagopal, Tamil Nadu (IN)

Correspondence Address: COOPER & DUNHAM, LLP, 30 Rockefeller Plaza, 20th Floor, NEW YORK, NY, 10112, US

(21) Appl. No.: 11/989,712

(22) PCT Filed: Aug. 4, 2006
(86) PCT No.: PCT/IB2006/002139

§ 371 Date: Jun. 30, 2008

(30) Foreign Application Priority Data
Aug. 5, 2005 (IN) 1074/CHE/2005

(51) Int. Cl. A61K 31/5377; C07D 417/12; A61K 31/4439; C12N 5/00; C07D 277/20; A61K 31/427; C07D 277/62; A61K 31/428; C07D 413/14; C07D 401/02; A61K 31/4545; C07D 401/14; C07D 333/22; A61K 31/381; C07C 271/02; A61K 31/27; C07D 235/04; A61K 31/4184; C07D 249/18; A61K 31/4192; C07D 215/38; A61K 31/47; C07D 405/02; A61K 31/496; C07D 403/02; A61P 35/00; A61P 17/06

(52) U.S. Cl. 514/236.8; 546/270.7; 514/342; 435/375; 548/196; 514/371; 548/179; 514/367; 544/131; 546/193; 514/318; 546/187; 514/316; 549/77; 514/438; 560/29; 514/487; 548/309.7; 514/394; 548/261; 514/359; 546/175; 514/311; 544/377; 514/254.11; 544/295; 514/252.14

(57) ABSTRACT

The present invention relates to novel compounds of the general formula (I), their derivatives, their analogs, their stereoisomers, their pharmaceutically acceptable salts and compositions. The present invention more particularly provides novel heterocyclic compounds of the general formula (I).

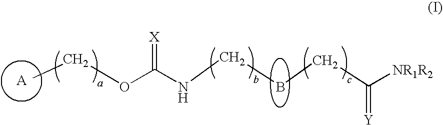

### FIELD OF THE INVENTION

The present invention relates to novel compounds of the general formula (I), their derivatives, their analogs, their stereoisomers, their pharmaceutically acceptable salts and compositions. The present invention more particularly provides novel heterocyclic compounds of the general formula (I).

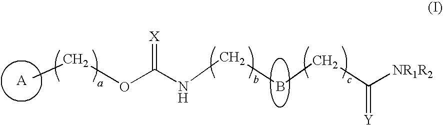

The present invention also provides a process for the preparation of the above said novel compounds of the formula (I), their derivatives, their analogs, their stereoisomers, their pharmaceutically acceptable salts and compositions.

The novel compounds (1) of the present invention are useful for the treatment cancer, which is one of the leading causes of death in the present society. A great deal of effort has been underway to treat various forms of cancer for decades and until recently; chemo prevention of cancer is receiving its due share of attention.

The first isolation of histone deacetylase was described in 1964 from crude nuclear extracts of cells, but the molecular characterization of isoforms of the enzyme has been achieved recently. Inhibitors of histone deacetylase (HDAC's) are zinc hydrolase's responsible for the deacetylation of N-acetyl lysine residues of historic and nonhistone protein substrates. Human HDAC's are classified into

two distinct classes, the HDAC's and sirtuins. The HDAC's are devised into two subclasses based on their similarity to yeast histone deacetylases, RPD 3 (class I includes HDAC 1, 2, 3, 8, and 11) and Hda 1 (class II includes HDAC 4, 6, 7, 9, and 10). All of the HDAC's have a highly conserved zinc dependent catalytic domain. There is growing evidence that the acetylation state of proteins and thus HDAC enzyme family plays a crucial role in the modulation of a number of biological processes, including transcription and the cell cycle.

Transcriptional regulation is a major event in cell differentiation, proliferation and apoptosis. Transcriptional activation of a set of genes determines cell destination and for this reason transcription is tightly regulated by a variety of factors. One of its regulatory mechanisms involved in the process is an alteration in the tertiary structure of DNA, which affects transcription factors to their target DNA regiments. Nucleosomal integrity is regulated by the acetylating status of the core histone, with the result being permissiveness to transcription. The acetylating status of the histone is governed by the balance of the activities of the histone acetyl transferase (HAT) and histone deacetylase (HDAC). Recently HDAC inhibitors have been found to arrest growth and apoptosis in several types of cancer cells, including colon cancer, t-cell lymphoma and erythroleukemic cells.

Given that apoptosis is a crucial factor for cancer progression, HDAC inhibitors are promising reagents for cancer therapy as effective inducers of apoptosis.

Several structural classes of HDAC inhibitors have been identified and are reviewed in Marks, P. A. et al., J. Natl. Cancer Inst., 92, (2000), 1210-1215. More specifically WO 98/55449 and U.S. Pat. No. 5,369,108 report alkanoyl hydroxamates with HDAC inhibitory activity.

### BACKGROUND OF THE INVENTION

The present invention relates to potentially pharmaceutical compositions and in particular to new molecules as active ingredients, that are used in particular as anticancer agents. Compounds of the general formula (I) or pharmaceutically acceptable salts thereof according to the present invention have an ability of inhibiting histone deacetylating enzyme and of inducing differentiation and are useful as therapeutic or ameliorating agent for diseases that are involved in cellular growth such as malignant tumors, autoimmune diseases, skin diseases, infections etc.

Few Prior Art References, which Disclose the Closest Compounds, are Given Here:
I. U.S. Pat. No. 6,638,530 D1 discloses benzamide derivatives of the formula (I)

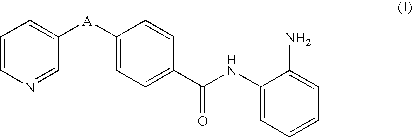

wherein A represents a structure represented by any one of the following

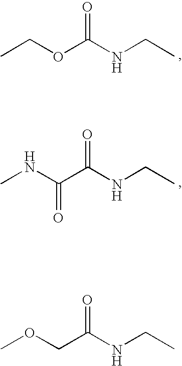

It is an object of the present invention to provide formulations with increased solubility and improved oral absorptivity, for benzamide derivatives of the formula (I) and their pharmaceutically acceptable salts that are useful as histone deacetylase inhibitors, and to provide injections containing the active ingredient at high concentrations.

II. U.S. Pat. No. 6,174,905 B1 discloses the compounds of the formula (I), wherein A, X, Q, R₁, R₂ and R₃ are as described thereof.

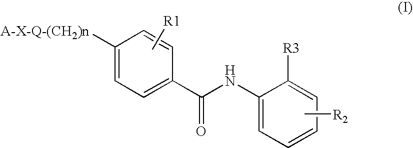

The novel benzamide derivative represented by formula (I) of this invention has differentiation-inducing effect, and are, therefore, useful as a therapeutic or improving agent for malignant tumors, autoimmune diseases, dermatologic diseases and parasitism. In particular, they are highly effective as an anticancer drug, specifically to a hematological malignancy and a solid carcinoma.

### OBJECTIVE OF THE INVENTION

Due to unmet medical needs and also as all of us know, cancer is one of the leading causes of death in the present society, we focused our attention to identify novel small molecule anticancer agents, particularly focusing on HDAC inhibitors. Our sustained efforts have resulted in novel anticancer agents of the formula (I). Histone acetylation and deacetylation play an essential role in modifying chromatin structure and regulating gene expression in eukaryotic cells. Hyper acetylated histones are generally found in transcriptionally active genes and in transcriptionally silent regions of the genome. Key enzymes, which modify histone proteins and thereby regulate gene expression, are histone acetyl transferases (HATs) and histone deacetylases (HDACs). Compounds able to inhibit HDAC activity i.e. HDAC inhibitors such as Trichostatin A (TSA), Trapoxin (TPX), Suberoylanilide hydroxamic acid (SAHA), Sodium butyrate (NaB), Sodium valproate (VPA), Cyclic hydroxamic acid containing peptides (CHAPs), Depsipeptide FK-228 and MS-275 can de-repress these genes, resulting in antiproliferative effects in vitro and anti tumor effects in vivo.

### SUMMARY OF THE INVENTION

The present invention relates to novel substituted heterocyclic compounds of the general formula (I),

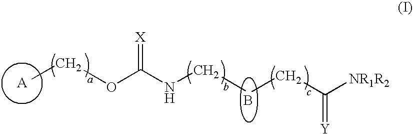

their derivatives, their analogs, their stereoisomers, their pharmaceutically acceptable salts and compositions, wherein A and B represent substituted or unsubstituted groups selected from aryl, aralkyl, heteroaryl and benzo fused heteroaryl; X and Y may be same or different and independently represent oxygen or sulphur or NR, wherein R represents hydrogen, hydroxy or an alkyl group; NR₁R₂, wherein R₁ and R₂ may be same or different and independently represent hydrogen, hydroxy, arylalkyl, carboxylic acid derivatives, alkyl, cycloalkyl, aryl, heteroaryl, alkoxy, benzyloxy, acetyl, benzyloxy acetyl or R₁ and R₂ may be fused to form a cyclic ring which may be selected from heterocyclyl, heteroaryl and benzo fused heteroaryl, all these groups may be further substituted; a, b and c are integers in the range of 0 to 2.

### DETAILED DESCRIPTION OF THE INVENTION

Suitable groups represented by A and B which may be substituted or unsubstituted groups are selected from aryl groups such as phenyl, naphthyl and the like; arylalkyl groups such as benzyl, phenylethyl, phenylpropyl and the like; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like; benzofused heteroaryl groups such as indolyl, indolinyl, benzodioxanyl, fluorenyl, benzimidazolyl, benzotriazolyl, benzothiazolyl, quinoline, quinoxaline, acridine, phenazine,

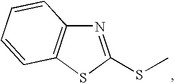

and the like. The point of attachment in case of the heteroaryl, heterocyclyl, and benzo fused heteroaryl rings to the remainder of the molecule may be through one of the hetero atoms or through carbon.

Suitable groups represented by X and Y which may be same or different and independently represent oxygen, sulphur or NR, wherein R represents hydrogen, hydroxy or alkyl groups such as methyl, ethyl, n-propyl, isopropyl, n-butyl, isobutyl, t-butyl and the like.

Suitable groups represented by NR₁R₂ wherein R₁ and R₂ may be same or different and independently represents hydrogen, hydroxyl, —CH₂COOEt, alkoxy groups such as methoxy, ethoxy, propoxy, n-butoxy, isobutoxy, t-butoxy and the like; benzyloxy, arylalkyl groups (such as benzyl, which may be substituted by one or more groups such as —OH, and the like), acetyl, trifluoro acetyl, benzyloxy acetyl, alkyl groups such as methyl, ethyl, n-propyl, isopropyl, n-butyl, isobutyl, t-butyl, n-pentyl and the like which may be substituted by one or more groups selected from alkoxy, hydroxy, substituted aryl, substituted benzyl, and —CO—NH-M, wherein M is —OH, —NO₂, —CH₂COOEt, haloalkyl, alkyl, alkenyl (such as ethenyl and the like), cycloalkyl, alkoxy and optionally substituted heteroaryl groups (for e.g., substituted with cycloalkyl); cycloalkyl groups such as cyclopropyl, cyclohexyl, cycloheptyl, cyclooctyl and the like which may be substituted; carboxylic acid derivatives (like esters, amides, and groups such as —O—(C═O)-M); aryl groups such as phenyl, naphthyl and the like, which may be substituted optionally by the groups selected from the following: —OH, —NH₂, —Ar*, —NH—CO-M, —NH—CO—Ar*, —OSO₂Me, —NH—CO—NH—Ar*, acylamino optionally substituted by S-M, wherein Ar* is selected from the groups such as phenyl, heteroaryl, heterocyclyl, and benzofused hetero aryl groups which are optionally substituted with —H, —OH, —CN and —OSO₂Me; wherein M is as described earlier; heterocyclyl groups such as pyrrolidinyl, thiazolidinyl, oxazolidinyl, morpholinyl, thiomorpholinyl, piperidinyl, piperazinyl, —NMethyl-piperazine and the like, which may be substituted by groups such as —(CH₂)ₑAr*, wherein Ar* is as described earlier; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like which may be substituted by the groups such as —NO₂, —CH₂COOEt, cycloalkyl (such as cyclopropyl and the like), haloalkyl groups (such as trifluoro methyl and the like), —(CH₂)_{g}—CO—NH-M, wherein M is as described earlier; benzofused heteroaryl groups such as indolyl, indolinyl, benzothiazolyl, quinoline, quinoxaline, acridine, phenazine and the like which may be substituted. e and f are integers in the range of 0 to 2.

R₁ and R₂ may be fused to form a cyclic ring, which may be selected from heterocyclyl groups such as pyrrolidinyl, imidazolidinyl, pyrazolidinyl, piperidinyl, morpholinyl, thiomorpholinyl, piperazinyl and the like which may be substituted by the groups such as alkyl, —CO—NH-M, wherein M is as described earlier, —(CH₂)_{g}Ar*, wherein Ar* is as described earlier or heteroaryl groups such as pyridyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like, which may be substituted or benzofused heteroaryl groups such as indolyl, indolinyl, benzothiazolyl, quinolinyl, quinoxalinyl, quinazolinyl, pteridinyl, acridinyl, phenazinyl, phenoxazinyl, phenothiazinyl, carbazolyl and the like which may be substituted. a, b, c and g are integers in the range of 0 to 2.

Suitable groups substituted (wherein the substitution may range from 1 position to all the available positions) on A and B may be selected from halogen (fluorine, chlorine, bromine, iodine), hydroxy, nitro, cyano, azido, nitroso, amino, hydrazine, formyl, alkyl, haloalkyl, haloalkoxy, cycloalkyl, aryl (may be further substituted), alkoxy, aryloxy, acyl, acyloxy, acyloxyacyl, heterocyclyl, heteroaryl (may be further substituted), monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups and carboxylic acids or its derivatives; wherein the definition of these groups remains same as defined earlier.

Furthermore when A and B are cyclic rings, they represent substituted or unsubstituted 5 to 10 membered ring systems, and also the rings may be monocyclic or bicyclic, saturated, partially saturated or aromatic, containing 1 to 4 hetero atoms selected from O, S and N and the like.

Pharmaceutically acceptable salts forming part of this invention include base addition salts such as alkali metal salts like Li, Na, and K salts, alkaline earth metal salts like Ca and Mg, salts of organic bases such as lysine, arginine, guanidine, diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising of other solvents of crystallization such as alcohols.

### Particularly Useful Compounds According to the Invention Include:

1. Pyridin-3-ylmethyl{4-[2-(hydroxyamino)-2-oxoethyl]-1,3-thiazol-2-yl}carbamate;
2. 2-Thienylmethyl{4-[2-(hydroxyamino)-2-oxoethyl]-1,3-thiazol-2-yl}carbamate;
3. 3-Thienylmethyl{4-[2-(hydroxyamino)-2-oxoethyl]-1,3-thiazol-2-yl}carbamate;
4. Pyridin-4-ylmethyl{4-[2-(hydroxyamino)-2-oxoethyl]-1,3-thiazol-2-4.yl}carbamate;
5. 2,3-Dihydro-1,4-benzodioxin-2-ylmethyl{4-[2-(hydroxyamino)-2-oxoethyl]1,3-thiazol-2-yl}carbamate;
6. (5-Bromo-2-thienyl)methyl{4-[2-(hydroxyamino)-2-oxoethyl]-1,3-thiazol-2-yl}carbamate;
7. (4-Bromo-2-thienyl)methyl{4-[2-(hydroxyamino)-2-oxoethyl]-1,3-thiazol-2-yl}carbamate;
8. (4-Methyl-1,3-thiazol-5-yl)methyl{4-[2-(hydroxyamino)-2-oxoethyl]-1,3-thiazol-2-yl}carbamate;
9. 1,3-Benzothiazol-2-ylmethyl{4-[2-(hydroxyamino)-2-oxoethyl]-1,3-thiazol-2-yl}carbamate;
10. Pyridin-3-ylmethyl[4-(2-morpholin-4-yl-2-oxoethyl)-1,3-thiazol-2-yl]carbamate
11. Pyridin-3-ylmethyl{4-[2-(1,3-benzothiazol-2-ylamino)-2-oxoethyl]-1,3-thiazol-2-yl}carbamate;
12. Pyridin-3-ylmethyl[4-(2-oxo-2-piperidin-1-ylethyl)-1,3-thiazol-2-yl]carbamate;
13. Pyridin-3-ylmethyl{4-[2-(1,4′-bipiperidin-1′-yl)-2-oxoethyl]-1,3-thiazol-2-yl}carbamate;
14. Pyridin-3-ylmethyl(4-{2-[methoxy(methyl)amino]-2-oxoethyl}-1,3-thiazol-2-yl)carbamate;
15. Pyridin-3-ylmethyl{4-[2-(methylamino)-2-oxoethyl]-1,3-thiazol-2-yl}carbamate;
16. Pyridin-3-ylmethyl{4-[2-(dimethylamino)-2-oxoethyl]-1,3-thiazol-2-yl}carbamate;
17. Pyridin-3-ylmethyl(4-{2-[(1-benzylpiperidin-4-yl)amino]-2-oxoethyl}-1,3-thiazol-2-yl)carbamate;
18. Pyridin-3-ylmethyl(4-{2-[(2-aminophenyl)amino]-2-oxoethyl}-1,3-thiazol-2-yl)carbamate;
19. Pyridin-3-ylmethyl(4-{2-[(2-hydroxyphenyl)amino]-2-oxoethyl}-1,3-thiazol-2-yl)carbamate;
20. Pyridin-3-ylmethyl(4-{2-[(3-hydroxyphenyl)amino]-2-oxoethyl}-1,3-thiazol-2-yl)carbamate;
21. 1,3-Benzothiazol-2-ylmethyl(4-{2-[(2-aminophenyl)amino]-2-oxoethyl}-1,3-thiazol-2-yl)carbamate;
22. Pentafluorobenzyl(4-{2-[(2-aminophenyl)amino]-2-oxoethyl}-1,3-thiazol-2-yl)carbamate;
23. 1,3-Thiazol-2-ylmethyl(4-{2-[(2-aminophenyl)amino]-2-oxoethyl}-1,3-thiazol-2-yl)carbamate;
24. Pyridin-4-ylmethyl(4-{2-[(2-aminophenyl)amino]-2-oxoethyl}-1,3-thiazol-2-yl)carbamate;
25. Pyridin-4-ylmethyl(4-{2-[(benzyloxy)amino]-2-oxoethyl}-1,3-thiazol-2-yl)carbamate;
26. Pentafluorobenzyl(4-{2-[(5-nitro-1,3-thiazol-2-yl)amino]-2-oxoethyl}-1,3-thiazol-2-yl)carbamate;
27. 3-Thienylmethyl{4-[(hydroxyamino)carbonyl]benzyl}carbamate;
28. 2-Thienylmethyl{4-[(hydroxyamino)carbonyl]benzyl}carbamate;
29. Pentafluorobenzyl{4-[(hydroxyamino)carbonyl]benzyl}carbamate;
30. 1,3-Benzothiazol-2-ylmethyl{4-[(hydroxyamino)carbonyl]benzyl}carbamate;
31. 1,3-Thiazol-2-ylmethyl{4-[(hydroxyamino)carbonyl]benzyl}carbamate;
32. 2,3-Dihydro-1,4-benzodioxin-2-ylmethyl {4-[(hydroxyamino)carbonyl]benzyl}carbamate;
33. 1H-Benzimidazol-2-ylmethyl{4-[(hydroxyamino)carbonyl]benzyl}carbamate;
34. 1H-1,2,3-Benzotriazol-1-ylmethyl{4-[(hydroxyamino)carbonyl]benzyl}carbamate;
35. 4-(Trifluoromethyl)benzyl{4-[(hydroxyamino)carbonyl]benzyl}carbamate;
36. 3,4,5-Trimethoxybenzyl{4-[(hydroxyamino)carbonyl]benzyl}carbamate;
37. Quinolin-4-ylmethyl{4-[(hydroxyamino)carbonyl]benzyl}carbamate;
38. 2,4,6-Trifluorobenzyl{4-[(hydroxyamino)carbonyl]benzyl}carbamate;
39. 2-(1,3-Benzothiazol-2-ylthio)ethyl{4-[(hydroxyamino)carbonyl]benzyl}carbamate;
40. 2-Thienylmethyl{4-[(methylamino)carbonyl]benzyl}carbamate;
41. 1H-Benzimidazol-2-ylmethyl{4-[(methylamino)carbonyl]benzyl}carbamate;
42. 2,3-Dihydro-1,4-benzodioxin-2-ylmethyl{4-[(methylamino)carbonyl]benzyl}carbamate;
43. 2-Thienylmethyl{4-[(methoxyamino)carbonyl]benzyl}carbamate;
44. Pentafluorobenzyl(4-{[methoxyamino]carbonyl}benzyl)carbamate;
45. 2,4,6-Trifluorobenzyl(4-{[hydroxy(methyl)amino]carbonyl}benzyl)carbamate;
46. 2-Thienylmethyl(4-{[hydroxy(methyl)amino]carbonyl}benzyl)carbamate;
47. Tentafluorobenzyl(4-{[hydroxy(methyl)amino]carbonyl}benzyl)carbamate;
48. 2-Thienylmethyl(4-{[methoxy(methyl)amino]carbonyl}benzyl)carbamate;
49. 3-Thienylmethyl(4-{[(2-aminophenyl)amino]carbonyl}benzyl)carbamate;
50. 3-Thienylmethyl{4-[(4-methylpiperazin-1-yl)carbonyl]benzyl}carbamate;
51. 3-Thienylmethyl[4-(morpholin-4-ylcarbonyl)benzyl]carbamate;
52. 2-Thienylmethyl(4-{[4-(1,3-benzodioxol-5-ylmethyl)piperazin-1-yl]carbonyl}benzyl)carbamate;
53. 2-Thienylmethyl (4-{[(2,2-dimethoxyethyl)amino]carbonyl}benzyl)carbamate;
54. Ethyl{2-[(4-{[(2-thienylmethyloycarbonyl)amino]methyl}benzoyl)amino]-1,3-thiazol-4-yl}acetate;
55. 2-Thienylmethyl(4-{[(3-aminophenyl)amino]carbonyl}benzyl)carbamate;
56. Pentafluorobenzyl(4-{[(5-cyclopropyl-1,3,4-thiadiazol-2-yl)amino]carbonyl}benzyl)carbamate;
57. 1,3-Thiazol-2-ylmethyl(4-{[4-(1,3-benzodioxol-5-ylmethyl)piperazin-1-yl]carbonyl}benzyl)carbamate;
58. 1,3-Benzothiazol-2-ylmethyl[4-({[5-(trifluoromethyl)-1,3,4-thiadiazol-2-yl]amino}carbonyl)benzyl]carbamate;
59. 2-Thienylmethyl[4-({[5-(trifluoromethyl)-1,3,4-thiadiazol-2-yl]amino}carbonyl)benzyl]carbamate;
60. 2,3-Dihydro-1,4-benzodioxin-2-ylmethyl[{4-(piperidinopiperidin-1-yl)carbonyl}benzyl]carbamate;
61. 2-Thienylmethyl(4-{[(2-hydroxyethyl)amino]carbonyl}benzyl)carbamate;
62. 2,4,6-Trifluorobenzyl(4-{[4-(1,3-benzodioxol-5-ylmethyl)piperazin-1-yl]carbonyl}benzyl)carbamate;
63. 2,4,6-Trifluorobenzyl(4-{[(5-cyclopropyl-1,3,4-thiadiazol-2-yl)amino]carbonyl}benzyl)carbamate;
64. 1,3-Thiazol-2-ylmethyl[4-({[5-(trifluoromethyl)-1,3,4-thiadiazol-2-yl]amino}carbonyl)benzyl]carbamate;
65. 1,3-Thiazol-2-ylmethyl(4-{[(5-cyclopropyl-1,3,4-thiadiazol-2-yl)amino]carbonyl}benzyl)carbamate;
66. 1,3-Benzothiazol-2-ylmethyl(4-{[4-(1,3-benzodioxol-5-ylmethyl)piperazin-1-yl]carbonyl}benzyl)carbamate;
67. 1,3-Benzothiazol-2-ylmethyl(4-{[(5-cyclopropyl-1,3,4-thiadiazol-2-yl) amino]carbonyl}benzyl)carbamate;
68. 1,3-thiazol-2-ylmethyl{4-[(4-pyrimidin-2-ylpiperazin-1-yl) carbonyl]benzyl}carbamate;
69. 2-thienylmethyl(4-{[(5-nitro-1,3-thiazol-2yl)amino]carbonyl}benzyl)carbamate;
70. 4-[(4-{[(2-thienylmethyloxycarbonyl)amino]methyl}benzoyl)amino]phenylmethanesulfonate;
71. 2-Thienylmethyl{4-[(4-pyridin-2-ylpiperazin-1-yl)carbonyl]benzyl}carbamate;
72. 2-Thienylmethyl{4-[(4-pyrimidin-2-ylpiperazin-1-yl) carbonyl]benzyl}carbamate;
73. 2-Thienylmethyl(4-{[(benzyloxy)amino]carbonyl}benzyl)carbamate;
74. Benzyl{4-[(hydroxyamino)carbonyl]pyridin-2-yl}carbamate;
75. Benzyl{5-[(hydroxyamino)carbonyl]-2-furyl}carbamate;
76. 8,8a-Dihydrocyclopenta[α]inden-8-ylmethyl{4-[(hydroxyamino)carbonyl]phenyl}carbamate;
77. 3-Thienylmethyl(4-{2-[acetyl(hydroxy)amino]-2-oxoethyl}-1,3-thiazol-2-yl)carbamate;
78. (5-bromo-2-thienyl)methyl(4-{2-[acetyl(hydroxy)amino]-2-oxoethyl}-1,3-thiazol-2-yl)carbamate;
79. (4-Bromo-2-thienyl)methyl(4-{2-[acetyl(hydroxy)amino]-2-oxoethyl}-1,3-thiazol-2-yl)carbamate;
80. 2-Thienylmethyl(4-{2-[acetyl(hydroxy)amino]-2-oxoethyl}-1,3-thiazol-2-yl)carbamate;
81. 2-Thienylmethyl{4-[({4-[(trifluoroacetyl)amino]phenyl}amino)carbonyl]benzyl}carbamate;
82. 2-Thienylmethyl(4-{[(acetyloxy)amino]carbonyl}benzyl)carbamate;
83. 1,3-thiazol-2-ylmethyl(4-{[(acetyloxy)amino]carbonyl}benzyl)carbamate;
84. 2-Thienylmethyl[4-({[(methoxycarbonyl)oxy]amino}carbonyl)benzyl]carbamate;
85. 2-Thienylmethyl[4-({[(methoxycarbonyl)oxy](methyl)amino}carbonyl)benzyl]carbamate;
86. 2-Thienylmethyl[4-({(N-benzyloxy)(N-methoxycarbonyl)amino}carbonyl)benzyl]carbamate;
87. Pyridin-3-ylmethyl[4-(2-{[6-(hydroxyamino)-6-oxohexyl]amino}-2-oxoethyl)-1,3-thiazol-2-yl]carbamate;
88. Pyridin-3-ylmethyl[4-(2-{[4-(hydroxyamino)-4-oxobutyl]amino}-2-oxoethyl)-1,3-thiazol-2-yl]carbamate;
89. (4-Methyl-1,3-thiazol-5-yl)methyl[4-(2-{[4-(hydroxyamino)-4-oxobutyl]amino}-2-oxoethyl)-1,3-thiazol-2-yl]carbamate;
90. 1,3-Benzothiazol-2-ylmethyl[4-(2-{[6-(hydroxyamino)-6-oxohexyl]amino}-2-oxoethyl)-1,3-thiazol-2-yl]carbamate;
91. 1,3-Benzothiazol-2-ylmethyl[4-(2-{[4-(hydroxyamino)-4-oxobutyl]amino}-2-oxoethyl)-1,3-thiazol-2-yl]carbamate;
92. Pentafluorobenzyl[4-(2-{[4-(hydroxyamino)-4-oxobutyl]amino}-2-oxoethyl)-1,3-thiazol-2-yl]carbamate;
93. 1,3-Benzothiazol-2-ylmethyl[4-(2-{[5-(hydroxyamino)-5-oxopentyl]amino}-2-oxoethyl)-1,3-thiazol-2-yl]carbamate;
94. Pentafluorobenzyl[4-(2-{[6-(hydroxyamino)-6-oxohexyl]amino}-2-oxoethyl)-1,3-thiazol-2-yl]carbamate;
95. 1,3-Thiazol-2-ylmethyl[4-(2-{[4-(hydroxyamino)-4-oxobutyl]amino}-2-oxoethyl)-1,3-thiazol-2-yl]carbamate;
96. 1,3-Thiazol-2-ylmethyl[4-(2-{[6-(hydroxyamino)-6-oxohexyl]amino}-2-oxoethyl)-1,3-thiazol-2-yl]carbamate;
97. Pyridin-4-ylmethyl[4-(2-{[4-(hydroxyamino)-4-oxobutyl]amino}-2-oxoethyl)-1,3-thiazol-2-yl]carbamate;
98. Pyridin-4-ylmethyl[4-(2-{[6-(hydroxyamino)-6-oxohexyl]amino}-2-oxoethyl)-1,3-thiazol-2-yl]carbamate;
99. 2-Thienylmethyl[4-({(2S)-2-[(hydroxyamino)carbonyl]pyrrolidin-1-yl}carbonyl)benzyl]carbamate;
100. 2-Thienylmethyl[4-({[2-(hydroxyamino)-1-(4-hydroxybenzyl)-2-oxoethyl]amino}carbonyl)benzyl]carbamate;
101. 2-Thienylmethyl{4-[({4-[2-(hydroxyamino)-2-oxoethyl]-1,3-thiazol-2-yl}amino)carbonyl]benzyl}carbamate;
102. 2-Thienylmethyl[4-({[6-(hydroxyamino)-6-oxohexyl]amino}carbonyl)benzyl]carbamate;
103. 2-Thienylmethyl[4-({[6-(5-cyclopropyl-1,3,4-thiadiazol-2-yl)-6-oxohexyl]amino}carbonyl)benzyl]carbamate;
104. Pyridin-4-ylmethyl[4-(2-{[2-({[(4-cyanophenyl)amino]carbonyl}-amino)phenyl]amino}-2-oxoethyl)-1,3-thiazol-2-yl]carbamate;
105. 2-Thienylmethyl[4-({[3-({[(4-cyanophenyl)amino]carbonyl}amino)phenyl]amino}carbonyl)benzyl]carbamate;
106. 2-Thienylmethyl(4-[({(2-{1,3-benzodioxol-5-ylcarbonyl}amino)-phenyl}amino)carbonyl]benzyl)carbamate;
107. 2-Thienylmethyl[4-({[4-(acryloylamino)phenyl]amino}carbonyl)benzyl]carbamate;
108. 2-Thienylmethyl[4-({[3-({[(difluoromethyl)thio]acetyl}amino)phenyl]amino}carbonyl)benzyl]carbamate;
109. Ethyl{methoxy[4-({[(2-thienylmethoxy)carbonyl]amino}-methyl)benzoyl]amino}acetate; and
110. 2-Thienylmethyl{4-[(E)-amino(hydroxyimino)-methyl]-phenyl}-carbamate;

According to another feature of the present invention, there is provided a process as shown in the following scheme, for the preparation of compounds of the formula (I), wherein all the groups are as defined earlier.
a) Condensation of the compound of formula (1a) and (1b) with 1,1′carbonyl imidazole yielded a compound of formula (1c), wherein A, B, a, and b are as defined earlier, and X is Oxygen.

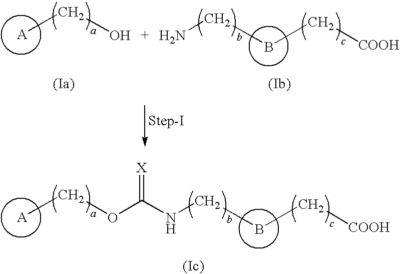

Furthermore the compound of formula (1c) is converted to the respective sulphur or N compound when X is S or NH by treatment with suitable reagents such as Lawesson's Reagent and the like.
b) Reaction of the compound of formula (1c) with an acid activating agent such as BOP, HOBt and the like in the presence of the respective amine HNR₁R₂ to yield the compound of the general formula (I) wherein R₁ and R₂ are as defined earlier and X may be O, S, or NH.

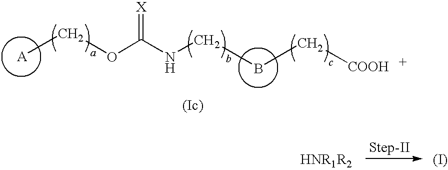

### The Compound of the General Formula (I) is Prepared by the Following Procedure:

Step (I): Condensation of the compound of formula (1a) and (1b) with 1,1′carbonyl imidazole is carried out in the presence of solvents selected from toluene, DMF, tetrahydrofuran, chloroform, dichloromethane, dichloroethane, ethyl acetate, o-dichlorobenzene or a mixture thereof, in the presence of bases such as triethylamine, diethylamine, pyridine, DMAP and alkali carbonates such as sodium carbonate, potassium carbonate and the like to afford the compound of formula (1c). The reaction is carried out at a temperature in the range of 0° C. to room temperature.

Step (II): The compound of formula (1c) is activated with acid activating reagents such as BOP, HOBt, DCC, isobutyl chloroformate and the like in the presence of solvents such as toluene, THF, DMF, chloroform, dichloromethane, dichloroethane, ethylacetate, o-dichlorobenzene or a mixture thereof, further reaction with hydroxylamine hydrochloride or the respective primary amine such as methylamine, aniline, 2-amino phenylamine and the like or secondary amines such as pyrrolidine, piperidine, N-methyl piperazine, morpholine, thiomorpholine and the like in the presence of a base such as triethylamine, diethylamine, diisopropyl ethylamine, pyridine, DMAP, alkali carbonates such as sodium carbonate, potassium carbonate and the like to produce a compound of formula (I). The reaction is carried out at a temperature in the range of 0° C. to room temperature for a time period ranging from 5 minutes to 10 hours.

In any of the above-mentioned reactions, any reactive group in the substrate molecule may be protected according to the conventional chemical practice. Suitable protecting groups in any of the above-mentioned reactions are those used conventionally in the art. The methods of formation and removal of such protecting groups are those conventional methods appropriate to the molecule being protected.

The pharmaceutically acceptable salts are prepared by reacting the compound of formula (I) with 1 to 4 equivalents of a base such as sodium hydroxide, sodium methoxide, sodium hydride, potassium t-butoxide, calcium

hydroxide, magnesium hydroxide and the like, in solvents like ether, THF, methanol, t-butanol, dioxane, isopropanol, ethanol etc. Mixtures of solvents may be used. Organic bases like lysine, arginine, diethanolamine, choline, guanidine and their derivatives etc. may also be used. Alternatively, acid addition salts are prepared by treatment with acids such as hydrochloric acid, hydrobromic acid, nitric acid, sulfuric acid, phosphoric acid, p-toluenesulfonic acid, methanesulfonic acid, acetic acid, citric acid, maleic acid, salicylic acid, hydroxynaphthoic acid, ascorbic acid, palmitic acid, succinic acid, benzoic acid, benzene sulfonic acid, tartaric acid and the like in solvents like ethyl acetate, ether, alcohols, acetone, THF, dioxane etc. Mixture of solvents may also be used.

In another aspect the invention provides novel pharmaceutical compositions comprising the heterocyclic derivatives of the formula (I) as set out above. The said compositions may comprise the heterocyclic derivatives as the active ingredient together with the pharmaceutically acceptable carrier, diluent or excipient. The composition may be prepared by processes known in the art and may be in the form of a tablet, capsule, powder, syrup, solution or suspension. The amount of the active ingredient in the composition may be less than 60% by weight.

The invention is explained in detail in the examples given below which are provided by the way of illustration only and therefore should not be construed to limit the scope of the invention.

### Example: 1

### Synthesis of pyridin-3-ylmethyl{4-[2-(hydroxyamino)-2-oxoethyl]-1,3-thiazol-2-yl}carbamate

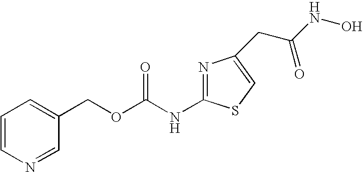

### Step: 1

### Preparation of methyl(2-amino-1,3-thiazol-4-yl)acetate

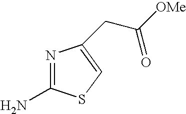

To a solution of 2-amino-4-thiazole acetic acid (20 g, 0.126 mol) in methanol (100 ml) was added concentrated H₂SO₄ (24 ml) dropwise at 0-5° C. and the reaction mixture was refluxed for 5 hours. Subsequently the reaction mixture was basified to pH of 8-9 with NaHCO₃ solution, to give a colorless precipitate, which was filtered and dried to afford the title compound in 19.5 g (93% yield).

### Step: 2

### Preparation of methyl(2-{[(pyridin-3-ylmethoxy)carbonyl]amino}-1,3-thiazol-4-yl)acetate

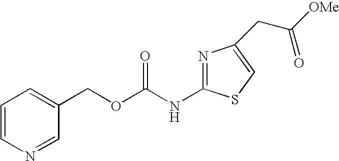

To a suspension of 1,1′-carbonylbis(1H-imidazole) (2.43 g, 15 mmol) in THF (12 mL) at 0-5° C. was added pyridine-3-methanol (1.63 g, 15 mmol) in THF (5 mL), and stirred at room temperature for 5 hours. The above reaction mixture was added to a solution of methyl (2-amino-1,3-thiazol-4-yl)acetate (2.58 g, 15 mmol), DBU (2.28 g, 15 mmol) and triethylamine (1.51 g, 15 mmol) in THF (24 ml) and stirred at room temperature, overnight. The THF was removed under reduced pressure and the crude compound was taken in dichloromethane (100 ml), washed with water, the organic layer dried over anhydrous Na₂SO₄, concentrated and purified by silica gel column chromatography using EtOAc/hexanes (4:6) to afford the title compound as a pale yellow colored solid (1 g, 23% yield).

### Step: 3

### Preparation of pyridin-3-ylmethyl{4-[2-(hydroxyamino)-2-oxoethyl]-1,3-thiazol-2-yl}carbamate

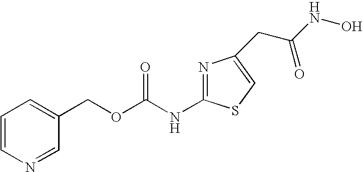

Hydroxylamine hydrochloride (1.25 g, 18 mmol) in methanol (3 mL) was mixed with KOH (1 g, 18 mmol) in methanol (6 mL) at 40° C., and cooled to 0° C., when a white precipitate was formed which was filtered. The filtrate was immediately added to (2-{[(pyridin-3-ylmethoxy)carbonyl]amino}-1,3-thiazol-4-yl)acetate (0.307 g, 1 mmol) followed by the addition of KOH (0.084 g, 1.5 mmol), and the mixture was stirred at room temperature, for 1 hour. Around 20 ml of water was added to the above and neutralized to a pH of 7 by dilute AcOH; on standing a colorless precipitate started forming which was filtered, dried and triturated with dichloromethane/hexanes (1:1) (20 mL), to afford the required compound as pure colorless solid (0.220 g, 71%) with m.p: 175-177° C. ¹H NMR (400 MHz, DMSO-d₆) δ (ppm): 3.32 (2H, s, —CH₂), 5.25 (2H, s, —OCH₂), 6.85 (1H, s, Ar—H), 7.45 (1H, s, Ar—H), 7.85 (1H, s, Ar—H), 8.54 (1H, s, Ar—H), 8.62 (1H, s, Ar—H), 9.01 (1H, s, D₂O exchangeable), 10.57 (1H, s, D₂O exchangeable), 11.89 (1H, s, D₂O exchangeable). MS m/z: 309 (M+1).

### Following Compounds are Prepared Following the Procedure Given in Example 1.

| Exp. | Structure | Analytical data |
|---|---|---|
| 2 | 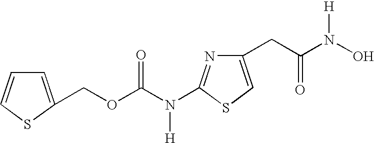 | ¹H NMR (DMSO-d₆) δ (ppm): 3.31 (2H, s, —CH₂), 5.38 (2H, s, —OCH₂), 6.88 (1H, s, Ar—H), 7.03-7.05 (1H, t, Ar—H), 7.22-7.23 (1H, d, Ar—H), 7.57-7.59 (1H, s, Ar—H), 8.83 (1H, s, D₂O exchangeable), 10.56 (1H, s, D₂O exchangeable), 11.77 (1H, s, D₂O exchangeable); M⁺ + 1 at 314; m.p: 155-157° C. |
| 3 | 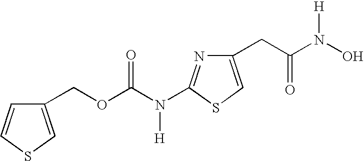 | ¹H NMR (DMSO-d₆) δ (ppm): 3.32 (2H, s, —CH₂), 5.21 (2H, s, —OCH₂), 6.84 (1H, s, Ar—H), 7.14-7.16 (1H, m, Ar—H), 7.54-7.58 (2H, m, Ar—H), 8.83 (1H, s, D₂O exchangeable), 10.57 (1H, s, D₂O echangeable), 11.75 (1H, s, D₂O exchangeable); M⁺ + 1 at 314; m.p: 157-161° C. |
| 4 | 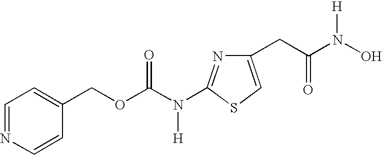 | ¹H NMR (DMSO-d₆) δ (ppm): 3.32 (2H, s, —CH₂), 5.27 (2H, s, —OCH₂), 6.87 (1H, s, Ar—H), 7.37-7.38 (2H, d, Ar—H), 8.57-8.58 (2H, d, Ar—H), 8.83 (1H, s, D₂O exchangeable), 10.57 (1H, s, D₂O exchangeable), 11.87 (1H, s, D₂O exchangeable); M⁺ − 1 at 307; m.p: 186- 189° C. |
| 5 | 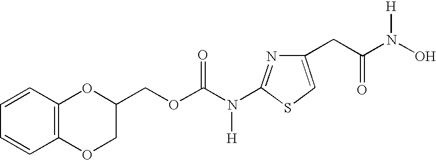 | ¹H NMR (DMSO-d₆) δ (ppm): 3.32 (2H, s, —CH₂), 4.04-4.09 (1H, dd, —OCH), 4.35- 4.46 (4H, m, —OCH₂), 6.82-6.90 (5H, m, Ar—H), 8.83 (1H, s,D₂O exchangeable), 10.57 (1H, s, D₂O exchangeable), 11.84 (1H, s, D₂O exchangeable); M⁺ + 1 at 366; m.p: 81-85° C. |
| 6 | 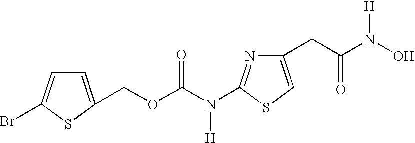 | ¹H NMR (DMSO-d₆) δ (ppm): 3.32 (2H, s, —CH₂), 5.32 (2H, s, —OCH₂), 6.89 (1H, s, Ar—H), 7.07-7.08 (1H, s, Ar—H), 7.14-7.15 (1H, d, Ar—H), 8.84 (1H, s, D₂O exchangeable), 10.56 (1H, s, D₂O exchangeable), 11.83 (1H, s, D₂O exchangeable); M⁺ + 1 at 393; m.p: 159-162° C. |
| 7 | 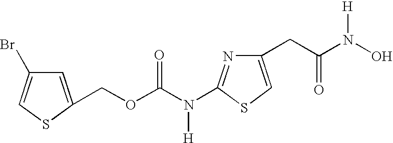 | ¹H NMR (DMSO-d₆) δ (ppm): 3.32 (2H, s, —CH₂), 5.34 (2H, s, —OCH₂), 6.87 (1H, s, Ar—H), 7.23 (1H, s, Ar—H) 7.66-7.67 (1H, d, Ar—H), 8.83 (1H, s, D₂O exchangeable), 9.96 (1H, s, D₂O exchangeable), 11.84 (1H, s, D₂O exchangeable); M⁺ + 1 at 393; m.p: 141-144° C. |
| 8 | 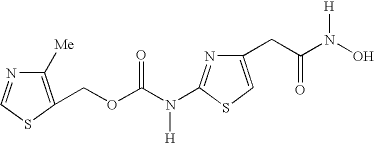 | ¹H NMR (DMSO-d₆) δ (ppm): 2.52 (3H, s, CH₃), 3.31 (2H, s,—CH₂), 5.41 (2H, s, —OCH₂), 6.88 (1H, s, Ar—H), 8.83 (1H, s, D₂O exchangeable), 9.01 (1H, s, Ar—H), 10.56 (1H, s, D₂O exchangeable) 11.80 (1H, s,D₂O exchangeable); M⁺ + 1 at 329; m.p: 176-179° C. |
| 9 | 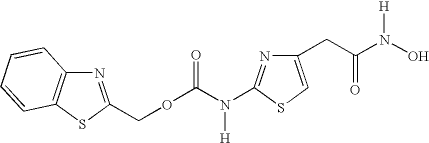 | ¹H NMR (DMSO-d₆) δ (ppm): 3.33 (2H, s, CH₂), 5.64 (2H, s, —OCH₂), 6.91 (1H, s, Ar—H), 7.47-7.50 (1H, t, Ar—H), 7.54-7.58 (1H, t, Ar—H), 8.02-8.04 (1H, d, Ar—H), 8.12-8.14 (1H, d, Ar—H), 8.85 (1H, s, D₂O exchangeable), 10.59 (1H, s, D₂O exchangeable), 12.15 (1H, s, D₂O exchangeable); M⁺ + 1 at 365; m.p: 171-175° C. |

### Example: 10

### Synthesis of pyridin-3-ylmethyl[4-(2-morpholin-4-yl-2-oxoethyl)-1,3-thiazol-2-yl]carbamate

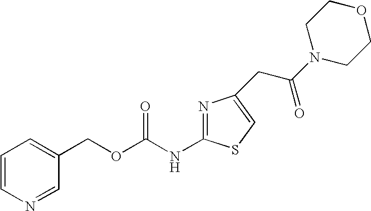

### Step: 1

### Preparation of (2-{[(pyridin-3-ylmethoxy)carbonyl]amino}-1,3-thiazol-4-yl)acetic acid

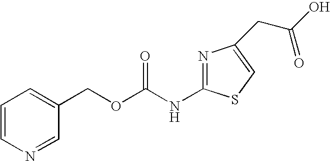

To a suspension of 1,1′-carbonylbis(1H-imidazole) (6.48 g, 40 mmol) in THF (30 ml) at 0-5° C. was added pyridine-3-methanol (4.36 g, 40 mmol) in THF (13 ml), and stirred at room temperature for 5 hours. The above reaction mixture was added to a suspension of 2-amino-4-thiazole acetic acid (6.37 g, 40 mmol), DBU (6.08 g, 40 mmol) and triethylamine (4.04 g, 40 mmol) in THF (63 ml) and stirred at room temperature, overnight. The THF was removed under reduced pressure and the crude compound was taken in dichloromethane (200 ml), washed with water, and the aqueous layer was acidified to a pH of 6 to afford a pale yellow colored precipitate which was filtered and dried to give the title compound (2.7 g, 25% yield).

### Step: 2

### Preparation of pyridin-3-ylmethyl[4-(2-morpholin-4-yl-2-oxoethyl)-1,3-thiazol-2-yl]carbamate

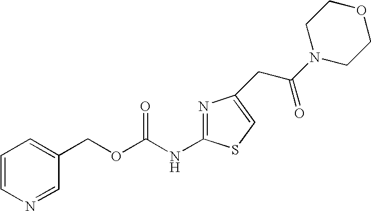

To a suspension of (2-{[(pyridin-3-ylmethoxy)carbonyl]amino}-1,3-thiazol-4-yl)acetic acid (0.293 g, 1 mmol) in THF (5 ml) was added DIPEA (0.4 mL, 2.4 mmol), EDCI (0.3 g, 1.6 mmol), HOBt (0.043 g, 0.32 mmol) followed by the morpholine (0.07 g, 0.8 mmol. The reaction was stirred at room temperature for 12 hours. The THF was removed under reduced pressure and the crude was taken up in EtOAc (50 ml) and washed with water, the organic layer was dried on anhydrous Na₂SO₄, concentrated and purified by silica gel column chromatography, using dichlormethane/MeOH (98:2) to afford 0.1 g (35% yield) of the title compound as pale yellow colored solid with m.p. 163-165° C. ¹H NMR (400 MHz, CDCl₃) δ (ppm): 3.50-3.51 (2H, d, —CH₂), 3.56-3.63 (2H, m, —CH₂), 3.72 (4H, s, —CH₂), 3.88 (2H, s, CH₂), 5.30 (2H, s, —OCH₂), 6.73 (1H, s, Ar—H), 7.36 (1H, s, Ar—H), 7.76-7.78 (1H, d, Ar—H), 8.65-8.71 (2H, d, Ar—H), 9.21 (1H, s, D₂O exchangeable). MS m/z: 363 (M+1).

### Following Compounds are Prepared Following the Procedure Given in Example: 10

| Exp. | Structure | Analytical data |
|---|---|---|
| 11 | 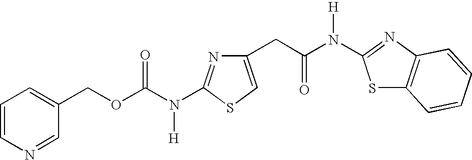 | ¹H NMR (DMSO-d₆) δ (ppm): 3.86 (2H, s, —CH₂), 5.26 (2H, s, —OCH₂), 7.03 (1H, s, Ar—H), 7.30- 7.43 (3H, d, Ar—H), 7.75-7.98 (3H, t, Ar—H), 8.55-8.64 (2H, d, Ar—H), 11.91 (1H, s, D₂O exchangeable), 12.53 (1H, s, D₂O exchangeable); M⁺ + 1 at 426; m.p: 218-220° C. |
| 12 | 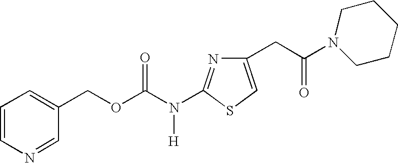 | ¹H NMR (CDCl₃) δ (ppm): 1.09- 1.14 (2H, t, —CH₂), 1.42-1.43 (2H, d, —CH₂), 1.58-1.59 (2H, d, CH₂), 3.35-3.38 (2H, t, —CH₂), 3.54-3.56 (2H, t, —CH₂), 3.71 (2H, s, —CH₂), 5.29 (2H, s, —OCH₂), 6.70 (1H, s, Ar—H), 7.31-7.35 (1H, m, Ar—H), 7.74-7.75 (1H, d, Ar—H), 8.62-8.63 (1H, d, Ar—H), 8.68 (1H, s, Ar—H), 9.85 (1H, s, D₂O exchangeable); M⁺ + 1 at 361; m.p: 127-130° C. |
| 13 | 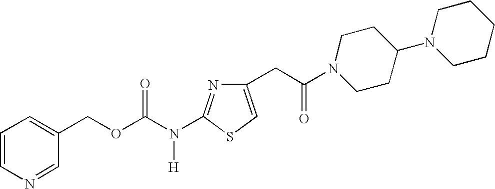 | ¹H NMR (DMSO-d₆) δ (ppm): 1.49 (2H, s, —CH₂), 1.70 (4H, s, —CH₂), 1.98-2.06 (3H, t, —CH₂), 2.35 (1H, s, —CH₂), 2.50-2.54 (2H, m, —CH₂), 2.85 (4H, s, —CH₂), 3.06 (2H, s, —CH₂), 3.57-3.61 (1H, d, —CH₂), 3.82-3.93 (2H, d, —CH₂), 5.10 (2H, s, —OCH₂), 6.68 (1H, s, Ar—H), 7.18-7.24 (1H, m, Ar—H) 7.54-7.60 (1H, m, Ar—H), 7.77 (1H, s, D₂O exchangeable), 8.55-8.56 (2H, d, D₂O exchangeable); M⁺ + 1 at 444; m.p: 70-72° C. |
| 14 | 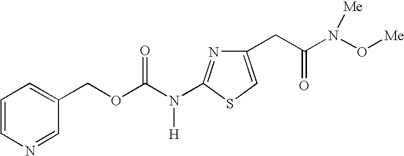 | ¹H NMR (CDCl₃) δ (ppm): 3.20 (3H, s, —CH₃) 3.65-3.69 (3H, s, —OCH₃), 3.80 (2H, s, —CH₂), 5.29- 5.30 (2H, d, —OCH₂), 6.76 (1H, s, Ar—H), 7.31-7.34 (1H, m, Ar—H), 7.72-7.75 (1H, m, Ar—H), 8.62-8.64 (1H, m, Ar—H), 8.68-8.69 (1H, d, Ar—H), 8.92 (1H, s, D₂O exchangeable); M⁺ + 1 at 337; m.p: 137-141° C. |
| 15 | 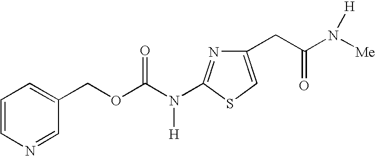 | ¹H NMR (DMSO-d₆) δ (ppm): 2.56- 2.57 (3H, d, —CH₃), 3.35-3.40 (2H, d, —CH₂), 5.26 (2H, s, —OCH₂), 6.87 (1H, s, Ar—H), 7.42-7.47 (1H, m, Ar—H) 7.84-7.86 (2H, d, Ar—H), 8.55-8.56 (1H, d, Ar—H), 8.63 (1H, s, D₂O exchangeable), 11.85 (1H, s, D₂O exchangeable). M⁺ + 1 at 307; m.p: 208-210° C. |
| 16 | 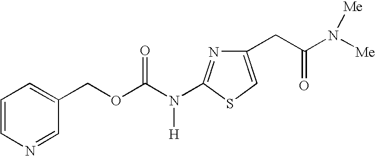 | ¹H NMR (CDCl₃) δ (ppm): 2.96 (3H, s, —CH₃), 3.03 (3H, s, —CH₃), 3.71 (2H, s, —CH₂), 5.28-5.30 (2H, d, —OCH₂), 6.73 (1H, s, Ar—H), 7.31- 7.35 (2H, m, Ar—H), 7.73-7.75 (1H, d, Ar—H), 8.61-8.63 (1H, d, Ar—H), 8.67 (1H, s, D₂O exchangeable); M⁺ + 1 at 321; m.p: 155-157° C. |
| 17 | 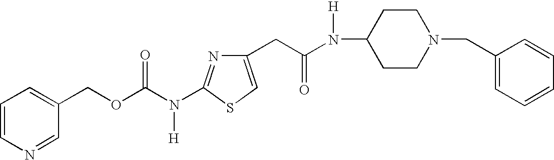 | ¹H NMR (DMSO-d₆) δ (ppm): 1.36- 1.41 (2H, t, —CH₂), 1.68-1.70 (2H, d, —CH₂), 1.98-2.08 (2H, d, —CH₂), 2.71 (2H, s, —CH₂), 3.32 (2H, s, —CH₂), 3.39-3.43 (2H, d, —NCH₂), 3.51 (1H, s, —NCH), 5.26 (2H, s, —OCH₂), 6.83 (2H, s, Ar—H), 7.23-7.33 (4H, m, Ar—H), 7.83-7.90 (2H, m, Ar—H), 8.55-8.56 (1H, d, Ar—H), 8.64 (1H, s, Ar—H), 11.82 (2H, s, D₂O exchangeable); M⁺ + 1 at 466; m.p: 200-202° C. |
| 18 | 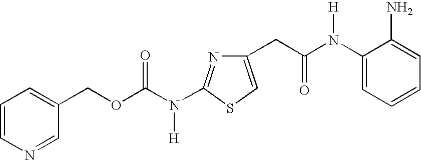 | ¹NMR (DMSO-d₆) δ (ppm): 3.66 (2H, s, —CH₂), 4.84 (2H, s, D₂O exchangeable), 5.27 (2H, s, —OCH₂), 6.50-6.54 (1H, t, Ar—H), 6.70-6.72 (1H, d, Ar—H), 6.87-6.91 (1H, t, Ar—H), 6.96 (1H, s, Ar—H), 7.15-7.17 (1H, d, Ar—H), 7.42-7.45 (1H, dd, Ar—H), 7.84-7.86 (1H, d, Ar—H), 8.55-8.56 (1H, d, Ar—H), 8.65 (1H, s, Ar—H), 9.27 (1H, s, D₂O exchangeable), 11.88 (1H, s, D₂O exchangeable); M⁺ + 1 at 384; m.p: 184-186° C. |
| 19 | 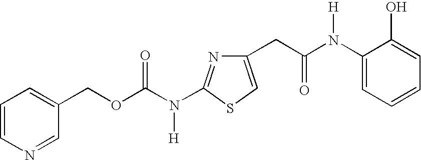 | ¹H NMR (DMSO-d₆) δ (ppm): 3.75 (2H, s, —CH₂), 5.27 (2H, s, —OCH₂), 6.75-6.78 (1H, t, Ar—H), 6.84-6.86 (1H, d, Ar—H), 6.91-6.96 (1H, m, Ar—H), 7.01 (1H, s, Ar—H), 7.43-7.46 (1H, m, Ar—H), 7.82-7.86 (2H, t, Ar—H), 8.55-8.56 (1H, d, Ar—H), 8.64 (1H, s, Ar—H), 9.21 (1H, s, D₂O exchangeable), 9.82 (1H, s, D₂O exchangeable), 11.92 (1H, s, D₂O exchangeable); M⁺ + 1 at 385; m.p: 205-208° C. |
| 20 | 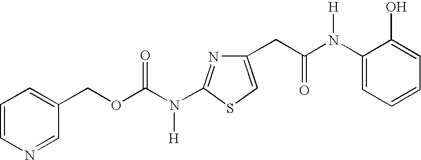 | ¹H NMR (DMSO-d₆) δ (ppm): 3.65 (2H, s, —CH₂), 5.26 (2H, s, —OCH₂), 6.42-6.44 (1H, m, Ar—H), 6.94-6.95 (2H, d, Ar—H), 7.03-7.07 (1H, t, Ar—H), 7.17 (1H, s, Ar—H), 7.41-7.52 (1H, m, Ar—H), 7.83-7.85 (1H, d, Ar—H), 8.55-8.56 (1H, d, Ar—H), 9.37 (1H, s, Ar—H), 9.99 (1H, s, D₂O exchangeable), 10.29 (1H, s, D₂O exchangeable), 11.87 (1H, s, D₂O exchangeable); M⁺ + 1 at 385; m.p: 208-211° C. |
| 21 | 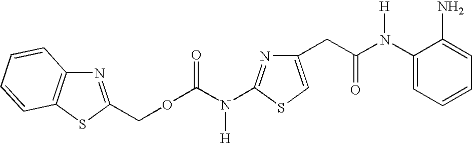 | ¹H NMR (DMSO-d₆) δ (ppm): 3.69 (2H, s, —CH₂), 4.86 (2H, s, D₂O exchangeable), 5.65 (2H, s, —OCH₂), 6.56-6.58 (1H, d, Ar—H), 6.72-6.74 (1H, d, Ar—H), 6.92-6.99 (1H, d, Ar—H), 7.15-7.17 (1H, d, Ar—H), 7.48 (1H, s, Ar—H), 7.50 (1H, s, Ar—H), 7.54-7.56 (1H, d, Ar—H), 8.02-8.04 (1H, d, Ar—H), 8.12-8.14 (1H, s, Ar—H), 9.29 (1H, s, D₂O exchangeable), 12.19 (1H, s, D₂O exchangeable); M⁺ + 1 at 440; m.p: 190-194° C. |
| 22 | 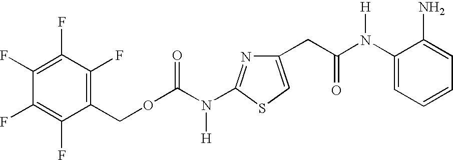 | ¹H NMR (DMSO-d₆) δ (ppm): 3.76 (2H, s, CH₂), 4.28 (2H, s, D₂O exchangeable), 5.38 (2H, s, —OCH₂), 6.39 (3H, s, Ar—H), 6.80 (1H, s, Ar—H), 7.22 (1H, s, Ar—H), 7.81 (1H, s, D₂O exchangeable), 8.41 (1H, s, D₂O exchangeable); M⁺ + 1 at 473; m.p: 142-147° C. |
| 23 | 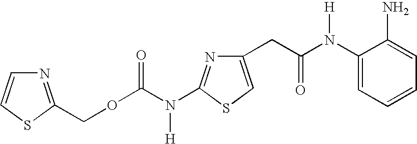 | ¹H NMR (DMSO-d₆) δ (ppm): 3.76 (2H, s, CH₂), 4.77 (2H, s, D₂O exchangeable), 5.57-5.59 (2H, d, —OCH₂), 6.76-6.81 (2H, m, Ar—H), 7.01-7.03 (1H, d, Ar—H), 7.30-7.32 (2H, d, Ar—H), 7.41-7.42 (2H, d, Ar—H), 7.83 (1H, s, D₂O exchangeable), 8.56 (1H, s, D₂O exchangeable); M⁺ 1 at 390; m.p: 160-164° C. |
| 24 | 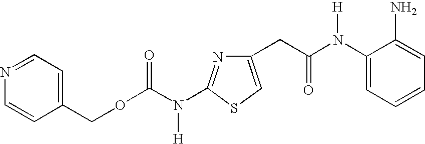 | ¹H NMR (DMSO-d₆) δ (ppm): 3.58 (2H, s, CH₂), 4.92 (2H, s, D₂O exchangeable), 5.17 (2H, s,—OCH₂), 6.50-6.54 (1H, m, Ar—H), 6.70-6.72 (2H, d, Ar—H), 6.86-6.90 (1H, m, Ar—H), 7.19-7.21 (1H, d, Ar—H), 7.31-7.36 (2H, d, Ar—H), 8.49-8.55 (2H, m, Ar—H), 9.44 (1H, s, D₂O exchangeable) 11.39 (1H, s, D₂O exchangeable); M⁺ + 1 at 384; m.p: 188-191° C. |
| 25 | 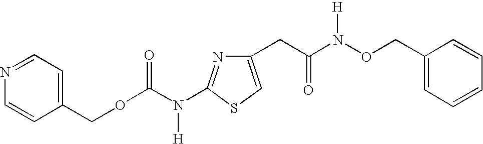 | ¹H NMR (DMSO-d₆) δ (ppm): 3.32 (2H, s, CH₂), 4.79 (2H, s, —OCH₂), 5.28 (2H, s, —OCH₂), 6.88 (1H, s, Ar—H), 7.36-7.40 (7H, m, Ar—H), 8.58-8.59 (2H, d, Ar—H), 11.20 (1H, s, D₂O exchangeable), 12.00 (1H, s, D₂O exchangeable); M⁺+ 1 at 399; m.p: 169.2-171.4° C. |
| 26 | 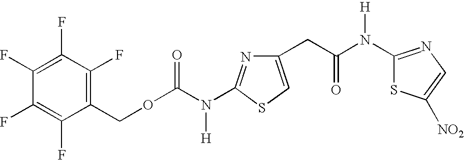 | ¹H NMR (DMSO-d₆) δ (ppm): 3.89 (2H, s, CH₂), 5.35 (2H, s, —OCH₂), 7.05 (1H, s, Ar—H), 8.64 (1H, s, Ar—H), 11.96 (1H, s, D₂O exchangeable), 13.28 (1H, s, D₂O exchangeable); M⁺ − 2 at 507; m.p: 219-223° C. |
| 27 | 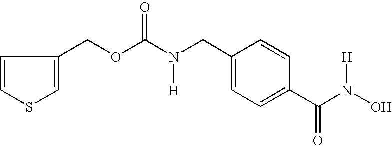 | ¹H NMR (DMSO-d₆) δ (ppm): 4.22- 4.24 (2H, d, —CH₂), 5.02 (2H, s, —CH₂), 7.10-7.11 (1H, d, Ar—H), 7.29-7.31 (2H, d, Ar—H), 7.49-7.53 (2H, t, Ar—H), 7.68-7.70 (2H, d, Ar—H), 7.83 (1H, t, D₂O exchangeable), 8.99 (1H, s, D₂O exchangeable), 11.16 (1H, s, D₂O exchangeable); M⁺ + 1 at 373; m.p: 163-164° C. |
| 28 | 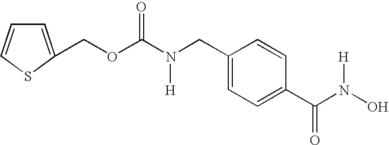 | ¹H NMR (DMSO-d₆) δ (ppm): 4.22- 4.24 (2H, d, —CH₂), 5.20 (2H, s, —CH₂), 7.00-7.02 (1H, dd, Ar—H), 7.13-7.14 (1H, d, Ar—H), 7.29-7.31 (2H, d, Ar—H), 7.53-7.54 (1H, t, Ar—H), 7.68-7.70 (2H, d, Ar—H), 7.85-7.89 (1H, t, D₂O exchangeable), 9.01 (1H, s, D₂O exchangeable), 11.17 (1H, s, D₂O exchangeable); M⁺ + 1 at 307.1; m.p: 143.5- 144.9° C. |
| 29 | 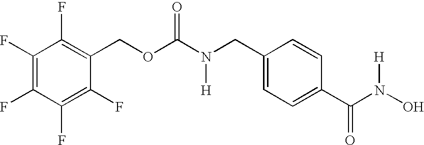 | ¹H NMR (DMSO-d₆) δ (ppm): 4.20- 4.22 (2H, d, —CH₂), 5.17 (2H, s, —CH₂), 7.28-7.30 (2H, d, Ar—H), 7.98 (1H, s, D₂O exchangeable), 9.01 (1H, s, D₂O exchangeable), 11.15 (1H, s, D₂O exchangeable); M⁺ − 1 at 389.0; m.p: 201.7-202.4° C. |
| 30 | 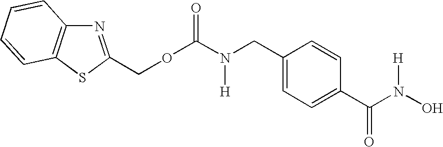 | ¹H NMR (DMSO-d₆) δ (ppm): 4.27- 4.29 (2H, d, —CH₂), 5.47 (2H, s, —CH₂), 7.33-7.35 (2H, d, Ar—H), 7.44-7.48 (1H, t, Ar—H), 7.52-7.56 (1H, dd, Ar—H), 7.70-7.72 (2H, d, Ar—H), 7.99-8.01 (1H, d, Ar—H), 8.12-8.14 (1H, d, Ar—H), 8.21-8.24 (1H, t, D₂O exchangeable), 9.02 (1H, s, D₂O exchangeable), 11.19 (1H, s, D₂O exchangeable); M⁺ + 1 at 358.0; m.p: 163.0-164.6° C. |
| 31 | 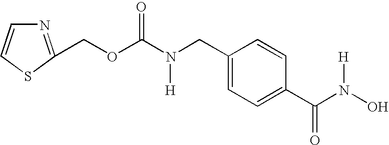 | ¹H NMR (DMSO-d₆) δ (ppm): 4.25- 4.26 (2H, d, —CH₂), 5.32 (2H, s, —CH₂), 7.31-7.33 (2H, d, Ar—H), 7.69-7.71 (2H, d, Ar—H), 7.75-7.81 (2H, dd, Ar—H), 8.10 (1H, t, D₂O exchangeable), 9.01 (1H, s, D₂O exchangeable), 11.18 (1H, s, D₂O exchangeable); M⁺+ 1 at 308.0; m.p: 136.0-139.1° C. |
| 32 | 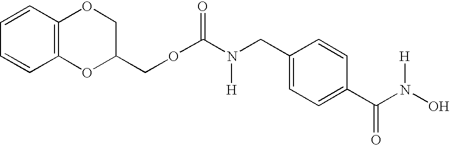 | ¹H NMR (DMSO-d₆) δ (ppm): 3.99- 4.03 (1H, m, CH) 4.18-4.28 (4H, m, —CH₂), 4.32-4.39 (2H, dd, —CH₂), 6.83-6.88 (4H, dd, Ar—H), 7.30-7.32 (2H, d, Ar—H), 7.69-7.70 (2H, d, Ar—H), 7.96-7.99 (1H, t, D₂O exchangeable), 9.01 (1H, s, D₂O exchangeable), 11.18 (1H, s, D₂O exchangeable); M⁺ + 1 at 359.1; m.p: 137.7-138.6° C. |
| 33 | 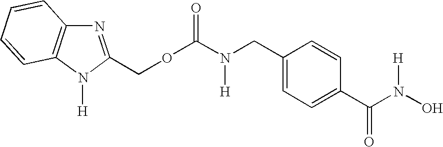 | ¹H NMR (DMSO-d₆) δ (ppm): 4.28 (2H, d, —CH₂), 5.27 (2H, s, —CH₂), 7.34-7.35 (1H, d, Ar—H), 7.41-7.43 (2H, d, Ar—H), 7.53-7.59 (3H, m, Ar—H), 7.69-7.73 (3H, t, Ar—H), 7.97-8.00 (1H, t, Ar—H), 8.00-8.02 (1H, t, D₂O exchangeable), 8.99 (1H, bs, D₂O exchangeable), 11.17 (1H, s, D₂O exchcangeable), 13.62 (1H, bs, D₂O exchangeable); M⁺ 1 at 341.2; m.p: 197.6-198.1° C. |
| 34 | 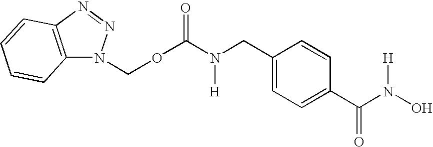 | ¹H NMR (DMSO-d₆) δ (ppm): 4.26- 4.30 (2H, d, —CH₂), 6.59-6.62 (1H, t, Ar—H), 6.66 (1H, d, Ar—H), 7.27-7.32 (2H, t, Ar—H), 7.35 (2H, s, CH₂), 7.61 (1H, s, D₂O exchangeable), 7.65-7.71 (2H, t, Ar—H), 7.89-7.90 (2H, d, Ar—H), 9.01 (1H, bs, D₂O exchangeable), 11.18 (1H, bs, D₂O exchangeable); M⁺ + 1 at 342.2; m.p: 243.1- 244.3° C. |
| 35 | 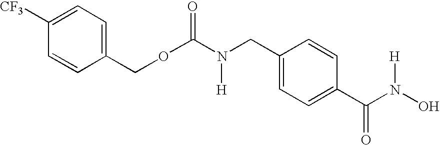 | ¹H NMR (DMSO-d₆) δ (ppm): 4.24- 4.25 (2H, d, CH₂), 5.15 (2H, s, CH₂), 7.30-7.33 (2H, d, Ar—H), 7.56-7.58 (2H, d, Ar—H), 7.69-7.71 (2H, d, Ar—H), 7.74-7.76 (2H, d, Ar—H), 7.98-8.01 (1H, t, Ar—H), 8.00-8.02 (1H, t, D₂O exchangeable), 8.99 (1H, bs, D₂O exchangeable), 9.09 (1H, s, D₂O exchangeable), 11.18 (1H, s, D₂O exchangeable); M⁺ − 1 at 366.8; m.p: 155.7-156.6° C. |
| 36 | 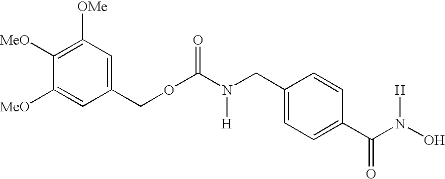 | ¹H NMR (DMSO-d₆) δ (ppm): 3.64 (3H, s, OCH₃), 3.75 (6H, s, OCH₃), 4.23-4.25 (2H, d, CH₂), 4.97 (2H, s, CH₂), 6.67 (2H, s, Ar—H), 7.30- 7.38 (2H, dd, Ar—H), 7.68-7.70 (2H, d, Ar—H), 7.88-7.91 (1H, t, D₂O exchangeable), 9.04 (1H, s, D₂O exchangeable), 11.18 (1H, s, D₂O exchangeable); M⁺ + 1 at 390.8; m.p: 65.3-67.1° C. |
| 37 | 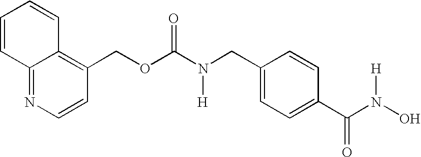 | ¹H NMR (DMSO-d₆) δ (ppm): 4.27- 4.28 (2H, d, CH₂), 5.61 (2H, s, CH₂), 7.33-7.35 (2H, d, Ar—H), 7.49-7.50 (1H, d, Ar—H), 7.70-7.77 (3H, d, Ar—H), 7.79-7.82 (1H, t, D₂O exchangeable), 9.03 (1H, s, Ar—H), 11.18 (1H, s, D₂O exchangeable); M⁺ + 1 at 352.0; m.p: 190.7- 191.4° C. |
| 38 | 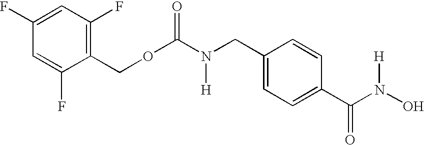 | ¹H NMR (DMSO-d₆) δ (ppm): 4.20- 4.22 (2H, d, CH₂), 5.06 (2H, s, CH₂), 7.20-7.24 (2H, t, Ar—H), 7.29-7.31 (2H, d, Ar—H), 7.68-7.70 (2H, d, Ar—H), 7.89-7.92 (1H, t, D₂O exchangeable), 9.01 (1H, bs, D₂O exchangeable), 11.14 (1H, bs, D₂O exchangeable); M⁺ + 1 at 355.2; m.p: 170.6-171.5° C. |
| 39 | 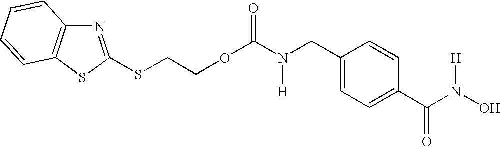 | ¹H NMR (DMSO-d₆) δ (ppm): 3.61- 3.64 (2H, t, CH₂), 4.21-4.22 (2H, d, CH₂), 4.33-4.36 (2H, t, CH₂), 7.30- 7.31 (2H, d, Ar—H), 7.35-7.39 (1H, t, Ar—H), 7.45-7.49 (1H, t, Ar—H) 7.68-7.70 (2H, d, Ar—H), 7.86-7.87 (1H, d, Ar—H), 7.88-7.89 (1H, t, D₂O exchangeable), 8.01-8.03 (1H, d, Ar—H), 9.01 (1H, s, Ar—H), 11.17 (1H, bs, D₂O exchangeable); M⁺ + 1 at 404.0; m.p: 145.4- 146.4° C. |
| 40 | 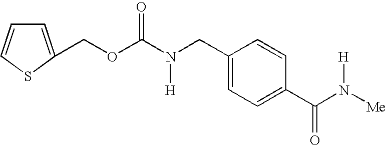 | ¹H NMR (DMSO-d₆) δ (ppm): 2.76- 2.77 (3H, d, CH₃), 4.23-4.25 (2H, d, CH₂), 5.20 (2H, s, CH₂), 7.00-7.02 (1H, q, Ar—H), 7.14-7.15 (1H, d, Ar—H), 7.31-7.33 (2H, d, Ar—H), 7.52-7.53 (1H, q, Ar—H), 7.75-7.77 (2H, d, Ar—H), 7.87-7.88 (1H, t, D₂O exchangeable), 8.37-8.38 (1H, d, D₂O exchangeable); M⁺ + 1 at 305.0; m.p: 152.8-153.7° C. |
| 41 | 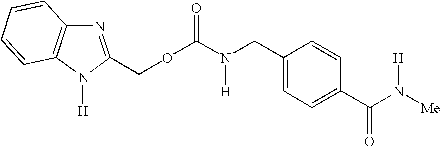 | ¹H NMR (DMSO-d₆) δ (ppm): 2.78 (3H, s, NCH₃), 4.27-4.28 (2H, d, CH₂), 5.21 (2H, s, CH₂), 7.21-7.27 (2H, q, Ar—H), 7.36-7.38 (2H, d, Ar—H), 7.51-7.53 (1H, d, Ar—H), 7.61-7.63 (1H, d, Ar—H), 7.77-7.79 (2H, d, Ar—H), 7.96-7.99 (1H, t, D₂O exchangeable), 8.38 (1H, s, D₂O exchangeable), 12.50 (1H, s, D₂O exchangeable); M⁺ + 1 at 339.2; m.p: 245.3-246.1° C. |
| 42 | 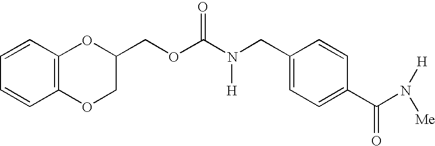 | ¹H NMR (DMSO-d₆) δ (ppm): 2.78- 2.79 (3H, d, NCH₃), 4.00-4.04 (1H, m, CH), 4.20-4.26 (4H, m, CH₂), 4.28-4.40 (2H, d, CH₂), 6.83-6.89 (4H, m, Ar—H), 7.31-7.33 (2H, d, Ar—H), 7.77-7.79 (2H, d, Ar—H), 7.963-7.99 (1H, t, D₂O exchangeable), 8.39-8.40 (1H, d, D₂O exchangeable). M⁺ + 1 at 356.9 m.p: 102.1-103.5° C. |
| 43 | 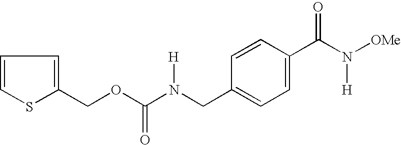 | ¹H NMR (DMSO-d₆) δ (ppm): 2.22 (3H, s, OCH₃), 4.25-4.27 (2H, d, CH₂), 5.20 (2H, s, CH₂), 7.00-7.02 (1H, t, Ar—H), 7.14-7.15 (1H, d, Ar—H), 7.36-7.38 (2H, d, Ar—H), 7.53-7.54 (1H, d, Ar—H), 7.74-7.75 (2H, d, Ar—H), 7.89-7.92 (1H, t, D₂O exchangeable), 12.27 (1H, s, D₂O exchangeable; M⁺ + 1 at 321.0; m.p: 158.1-159.5° C. |
| 44 | 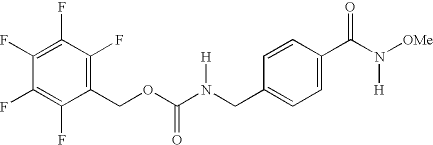 | ¹H NMR (DMSO-d₆) δ (ppm): 3.69 (3H, s, OCH₃), 4.21-4.22 (2H, d, CH₂), 5.16 (2H, s, CH₂), 7.29-7.31 (2H, d, Ar—H), 7.68-7.70 (2H, d, Ar—H), 7.98-8.01 (1H, t, D₂O exchangeable), 11.70 (1H, s, D₂O exchangeable); M⁺ + 1 at 405.0; m.p: 166.7-168.0° C. |
| 45 | 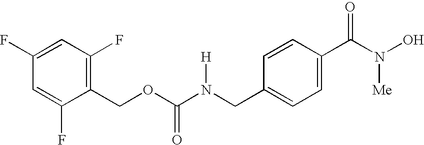 | ¹H NMR (DMSO-d₆), δ (ppm): 3.34 (3H, s, CH₃), 4.19-4.20 (2H, d, CH₂), 5.06 (2H, s, CH₂), 7.24-7.28 (4H, t, Ar—H), 7.54-7.56 (2H, d, Ar—H), 7.90-7.91 (1H, t, D₂O exchangeable), 9.97 (1H, s, D₂O exchangeable). M⁺ + 1 at 369.0; m.p: 116.0-118.1° C. |
| 46 | 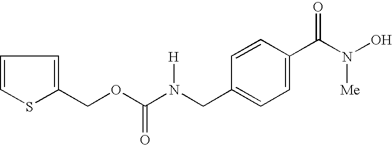 | ¹H NMR (DMSO-d₆) δ (ppm): 3.34 (3H, s, CH₃), 4.22-4.23 (2H, d, CH₂), 5.20 (2H, s, CH₂), 7.00-7.02 (1H, t, Ar—H), 7.14-7.15 (1H, d, Ar—H), 7.26-7.28 (2H, d, Ar—H), 7.53-7.56 (3H, t, Ar—H), 7.86-7.89 (1H, t, D₂O exchangeable), 9.98 (1H, s, D₂O exchangeable); M⁺ + 1 at 321.1; m.p: 131.6-132.7° C. |
| 47 | 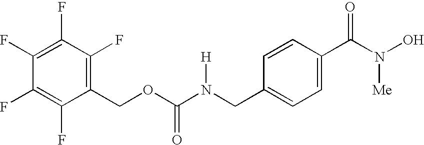 | ¹H NMR (DMSO-d₆) δ (ppm): 3.23 (3H, s, NCH₃) 4.19-4.20 (2H, d, CH₂), 5.17 (2H, s, CH₂), 7.24-7.26 (2H, d, Ar—H), 7.54-7.56 (2H, d, Ar—H), 7.98-7.99 (1H, t, D₂O exchangeable), 9.98 (1H, s, D₂O exchangeable). M⁺ − 1 at 403.0; m.p: 140.9-141.2° C. |
| 48 | 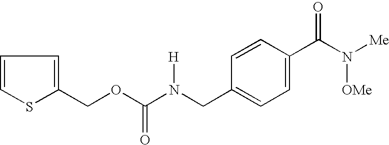 | ¹H NMR (DMSO-d₆) δ (ppm): 3.24 (3H, s, N—CH₃), 3.53 (3H, s, OCH₃), 4.24-4.25 (2H, d, CH₂), 5.21 (2H, s, CH₂), 7.00-7.02 (1H, t, Ar—H), 7.14-7.15 (1H, d, Ar—H), 7.30-7.31 (2H, d, Ar—H), 7.53-7.55 (3H, t, Ar—H), 7.86-7.89 (1H, t, D2O exchangeable); M⁺ + 1 at 335.0 |
| 49 | 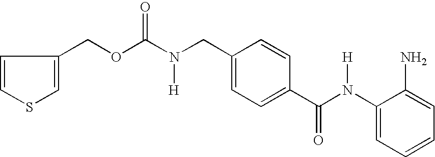 | ¹H NMR (DMSO-d₆) δ (ppm): 3.48 (2H, bs, D₂O exchangeable), 4.28- 4.29 (2H, d, CH₂), 5.04 (2H, s, CH₂), 7.05 (1H, s, Ar—H), 7.11-7.13 (2H, d, Ar—H), 7.19-7.20 (1H, d, Ar—H), 7.31-7.33 (1H, d, Ar—H), 7.40-7.42 (2H, d, Ar—H), 7.49-7.50 (2H, d, Ar—H), 7.88 (1H, s, D₂O exchangeable) 7.97-7.99 (2H, d, Ar—H), 10.06 (1H, s, D₂O exchangeable); M⁺ + 1 at 382.1; m.p: 135-137° C. |
| 50 | 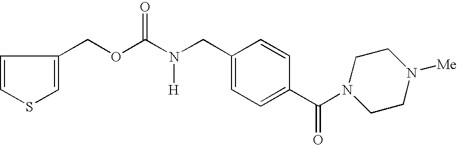 | ¹H NMR (DMS)-d₆) δ (ppm): 2.19 (3H, s, CH₃), 2.20-2.29 (4H, d, piperazine), 3.41-3.43 (2H, s, piperazine), 3.55-3.60 (2H, s, piperazine), 4.23-4.24 (2H, d, CH₂), 5.03 (2H, s, CH₂), 7.11-7.12 (1H, d, Ar—H), 7.30-7.31 (4H, d, Ar—H), 7.32-7.33 (2H, d, Ar—H) 7.83 (1H, t, D₂O exchangeable); M⁺ + 1 at 382.1 |
| 51 | 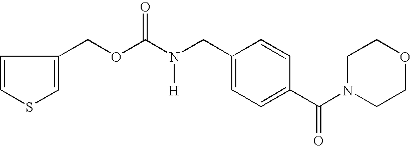 | ¹H NMR (DMSO-d₆) δ (ppm): 3.3 (4H, bs, piperazine), 3.6 (4H, bs, piperazine), 4.22-4.24 (2H, d, CH₂), 5.03 (2H, s, CH₂), 7.10-7.11 (1H, d, Ar—H), 7.30-7.32 (2H, d, Ar—H), 7.35-7.37 (2H, d, Ar—H), 7.48 (1H, s, Ar—H), 7.52-7.54 (1H, t, Ar—H), 7.82-7.85 (1H, t, D₂O exchangeable); M⁺ + 1 at 382.1 |
| 52 | 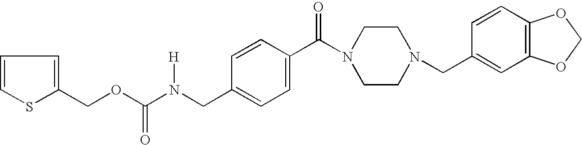 | ¹H NMR (DMSO-d₆) δ (ppm): 2.33- 2.38 (4H, d, CH₂), 3.32 (2H, s, CH₂), 3.42 (2H, s, CH₂), 3.60 (2H, s, CH₂), 4.22-4.24 (2H, d, CH₂), 5.20 (2H, s, CH₂), 5.79 (2H, s, CH₂), 6.76-6.78 (1H, q, Ar—H), 6.83-6.86 (2H, q, Ar—H), 7.01-7.02 (H, t, Ar—H), 7.13-7.14 (1H, d, Ar—H), 7.28-7.31 (4H, t, Ar—H), 7.52-7.54 (1H, q, Ar—H), 7.86-7.88 (1H, t, D2O exchangeable); M⁺ + 1 at 494.1 |
| 53 | 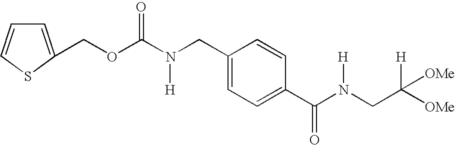 | ¹H NMR (DMSO-d₆) δ (ppm): 3.28 (6H, s, OCH₃), 3.36-3.38 (2H, d, CH₂), 4.24-4.25 (2H, d, CH₂), 4.50- 4.52 (1H, t, CH), 5.21 (2H, s, CH₂), 7.01-7.04 (1H, t, Ar—H), 7.15 (1H, s, Ar—H) 7.30-7.33 (2H, d, Ar—H), 7.53-7.54 (1H, d, Ar—H), 7.79-7.80 (2H, d, Ar—H), 7.87 (1H, s, D₂O exchangeable), 8.50 (1H, s, D₂O exchangeable). M⁺ + 1 at 379.1 m.p: 106.5-107.0° C. |
| 54 | 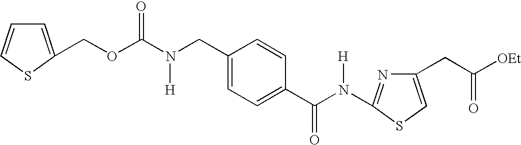 | ¹H NMR (DMSO-d₆) δ (ppm): 1.21 (3H, s, CH₃), 3.74 (2H, s, CH₂), 4.06-4.12 (2H, q, CH₂), 4.27-4.29 (2H, d, CH₂), 5.22 (2H, s, CH₂), 7.00-7.03 (1H, t, Ar—H), 7.05 (1H, s, Ar—H), 7.15-7.16 (1H, d, Ar—H), 7.37-7.39 (2H, d, Ar—H), 7.54-7.55 (1H, d, Ar—H), 7.90-7.94 (1H, t, D₂O exchangeable), 8.03-8.04 (2H, d, Ar—H), 12.64 (1H, s, D₂O exchangeable); M⁺ + 1 at 460.0 m.p: 96.3-100.6° C. |
| 55 | 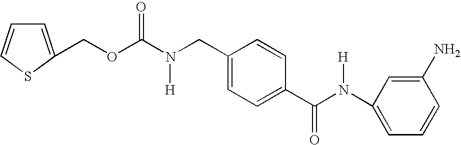 | ¹H NMR (DMSO-d₆) δ (ppm): 4.26- 4.28 (2H, d, CH₂), 5.08 (2H, s, D₂O exchangeable), 5.21 (2H, s, CH₂), 6.36-6.38 (1H, t, Ar—H), 6.84-6.86 (1H, d, Ar—H), 6.93-6.95 (H, d, Ar—H), 7.02-7.03 (1H, t, Ar—H), 7.09 (1H, s, Ar—H), 7.14-7.15 (1H, d, Ar—H), 7.35-7.37 (2H, d, Ar—H), 7.53-7.55 (1H, d, Ar—H), 7.84-7.86 (2H, d, Ar—H), 7.86-7.91 (1H, t, D₂O exchangeable), 9.89 (1H, s, D₂O exchangeable); M⁺ + 1 at 382.1. |
| 56 | 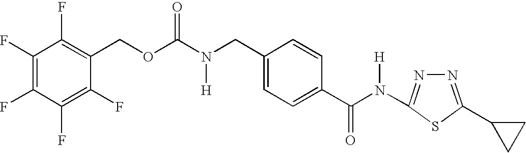 | ¹H NMR (DMSO-d₆) δ (ppm): 1.00- 1.01 (2H, t, cyclopropane), 1.15-1.16 (2H, t, cyclopropane₎, 2.33-2.38 (1H, m, CH), 4.26-4.27 (2H, d, CH₂), 5.18 (2H, s, CH₂), 7.38-7.40 (2H, d, Ar—H), 8.02-8.03 (2H, d, Ar—H), 8.04-8.05 (1H, t, D₂O exchangeable), 12.86 (1H, bs, D₂O exchangeable); M⁺ + 1 at 499.0. |
| 57 | 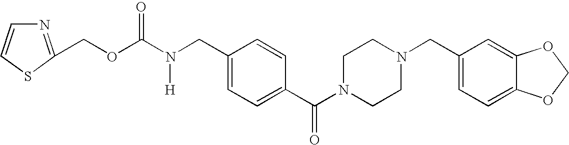 | ¹H NMR (DMSO-d₆) δ (ppm): 2.33- 2.36 (4H, bd, piper), 3.32-3.41 (4H, bd, piper), 3.57 (2H, s, CH₂), 4.25- 4.26 (2H, d, CH₂), 5.32 (2H, S, CH₂), 5.98 (2H, s, CH₂) 6.73-6.75 (1H, d, Ar—H), 6.83 (1H, s, Ar—H), 6.85-6.86 (1H, d, Ar—H), 7.30-7.34 (4H, d, Ar—H), 7.75-7.76 (1H, d, Ar—H), 7.80-7.81 (1H, d, Ar—H) 8.08-8.11 (1H, t, D₂O exchangeable); M⁺ + 1 at 495.1 |
| 58 | 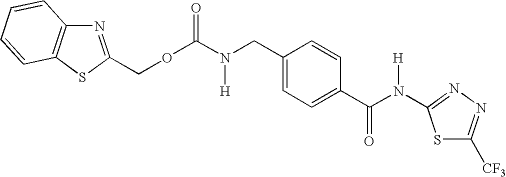 | ¹H NMR (DMSO-d₆) δ (ppm): 4.35- 4.36 (2H, d, CH₂), 5.49 (2H, s, CH₂), 7.45-7.56 (4H, m, Ar—H), 8.00-8.02 (1H, d, Ar—H), 8.12-8.14 (3H, d, Ar—H), 8.30 (1H, t, D₂O exchangeable), 13.72 (1H, s, D₂O exchangeable); M⁺ + 1 at 494.0; m.p: 241.6-243.1° C. |
| 59 | 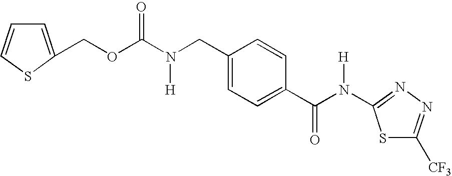 | ¹H NMR (DMSO-d₆) δ (ppm): 4.30- 4.32 (2H, d, CH₂), 5.22 (2H, s, CH₂), 7.01-7.03 (1H, q, Ar—H), 7.15-7.16 (1H, d, Ar—H), 7.43-7.46 (2H, d, Ar—H) 7.54-7.55 (1H, q, Ar—H), 7.93-7.96 (1H, t, D₂O exchangeable), 8.10-8.13 (2H, d, Ar—H), 13.72 (1H, s, D₂O exchangeable); M⁺ + 1 at 443.0; m.p: 231.7-232.6° C. |
| 60 | 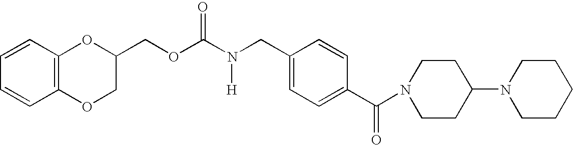 | ¹H NMR (DMSO-d₆) δ (ppm): 1.49 (2H, bs, CH₂), 1.70 (4H, bs, CH₂), 1.98-2.06 (3H, t, CH₂), 2.35 (1H, s, CH), 2.53 (1H, t, CH), 2.85 (4H, bs, CH₂), 3.06 (2H, t, CH₂), 3.57- 3.61 (1H, d, CH) 3.82-3.86 (1H, d, CH), 4.00-4.04 (1H, m, CH), 4.20- 4.26 (4H, m, CH₂), 4.35-4.36 (2H, d, CH₂), 6.83-6.90 (4H, m, Ar—H) 7.29-7.34 (4H, m, Ar—H) 7.95-7.98 (1H, t, D₂O exchangeable); M⁺ + 1 at 494.6 |
| 61 | 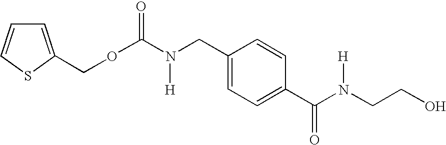 | ¹H NMR (DMSO-d₆) δ (ppm): 3.34- 3.37 (2H, t, CH₂), 3.52-3.55 (2H, t, CH₂) 4.23-4.25 (2H, d, CH₂), 4.73 (1H, s, D₂O exchangeable), 5.20 (2H, s, CH₂), 6.98-7.03 (1H, t, Ar—H) 7.12-7.13 (1H, d, Ar—H) 7.35-7.37 (2H, d, Ar—H), 7.50-7.51 (1H, d, Ar—H), 7.73-7.75 (2H, d, Ar—H), 7.89-7.90 (1H, t, D₂O exchangeable), 8.41-8.42 (1H, t, D₂O exchangeable); M⁺ + 1 at 334.9; m.p: 160.2-161.6° C. |
| 62 | 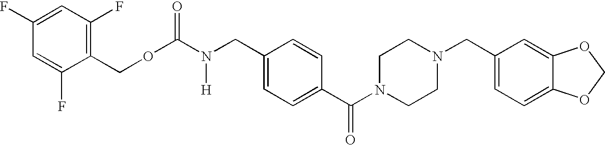 | ¹H NMR (DMSO-d₆) δ (ppm): 2.39- 2.53 (4H, bd, piperazine) 3.25-3.48 (4H, bd, piperazine), 3.60 (2H, s, CH₂), 4.21 (2H, s, CH₂), 5.07 (2H, s, CH₂), 5.98 (2H, s, CH₂), 6.74- 6.76 (1H, d, Ar—H), 6.83-6.86 (2H, t, Ar—H), 6.23-7.34 (6H, m, Ar—H), 7.89-7.92 (1H, t, D₂O exchangeable); M⁺ + 1 at 542.3; m.p: 68.9-70.7° C. |
| 63 | 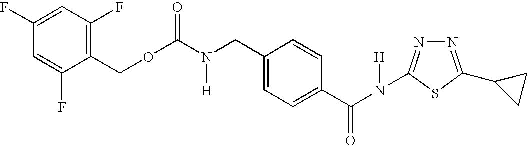 | ¹H NMR (DMSO-d₆) δ (ppm): 1.00- 1.01 (2H, t, cyclopropane), 1.13-1.16 (2H, t, cyclopropane₎, 2.39-2.45 (1H, m, CH), 4.26-4.27 (2H, d, CH₂), 5.07 (2H, s, CH₂), 7.25-7.29 (2H, t, Ar—H), 7.38-7.39 (2H, d, Ar—H), 7.95-7.97 (1H, t, D₂O exchangeable), 8.03-8.05 (2H, d, Ar—H), 12.87 (1H, s, D₂O exchangeable); M⁺ + 1 at 463.0; m.p: 271.2-271.8° C. |
| 64 | 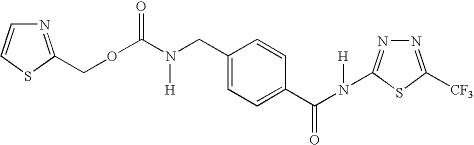 | ¹H NMR (DMSO-d₆) δ (ppm): 4.33- 4.35 (2H, d, CH₂), 5.35 (2H, s, CH₂), 7.46-7.48 (2H, d, Ar—H), 7.76 (1H, s, Ar—H), 7.82 (1H, s, Ar—H) 8.11-8.13 (2H, d, Ar—H), 8.17-8.20 (1H, t, D₂O exchangeable), 13.78 (1H, bs, D₂O exchangeable). M⁺ − 1 at 441.9; m.p: 301° C. (with decomposition). |
| 65 | 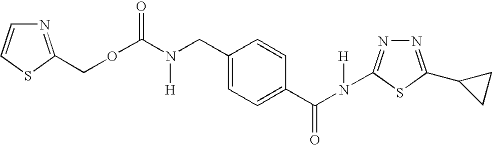 | ¹H NMR (DMSO-d₆) δ (ppm): 1.00- 1.02 (2H, t, cyclopropane), 1.13-1.16 (2H, t, cyclopropane), 2.41-2.43 (1H, m, CH), 4.31-4.32 (2H, d, CH₂) 5.34 (2H, s, CH₂), 7.41-7.43 (2H, d, Ar—H), 7.76-7.77 (1H, d, Ar—H), 7.81-7.82 (1H, d, Ar—H), 8.05-8.07 (2H, d, Ar—H), 8.15-8.18 (1H, t, D₂O exchangeable) 12.89 (1H, bs, D₂O exchangeable); M⁺ − 1 at 414.0; m.p: 213.6-215.3° C. |
| 66 | 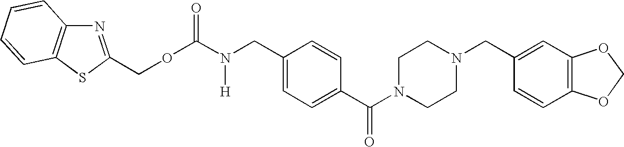 | ¹H NMR (DMSO-d₆) δ (ppm): 2.33- 2.39 (4H, bd, piperazine), 3.32-3.41 (4H, bd, piperazine), 3.60 (2H, s, CH₂), 4.27-4.28 (2H, d, CH₂), 5.46 (2H, s, CH₂), 5.98 (2H, s, CH₂) 6.76-6.78 (1H, d, Ar—H), 6.84-6.88 (2H, t, Ar—H), 7.35 (4H, s, Ar—H) 7.45-7.48 (1H, t, Ar—H), 7.53-7.57 (1H, t, Ar—H), 7.99-8.01 (1H, d, Ar—H), 8.10-8.12 (1H, d, Ar—H), 8.22-8.25 (1H, t, D₂O exchangeable); M⁺ + 1 at 545.1; m.p: 68.6-70.1° C. |
| 67 | 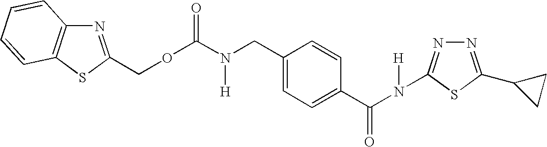 | ¹H NMR (DMSO-d₆) δ (ppm): 1.00- 1.02 (2H, t, cyclopropane), 1.14-1.16 (2H, t, cyclopropane), 2.40-2.44 (1H, m, CH), 4.34-4.35 (2H, d, CH₂) 5.49 (2H, s, CH₂), 7.44-7.49 (3H, t, Ar—H), 7.53-7.56 (1H, t, Ar—H), 8.00-8.02 (1H, d, Ar—H), 8.06-8.08 (2H, d, Ar—H), 8.13-8.15 (1H, d, Ar—H), 8.28-8.31 (1H, t, D₂O exchangeable) 12.88 (1H, bs, D₂O exchangeable). M⁺ + 1 at 466.0; m.p: 211.4-213.8° C. |
| 68 | 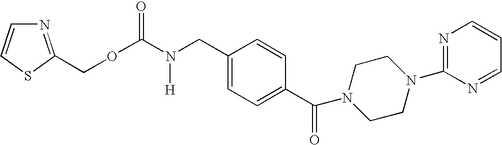 | ¹H NMR (DMSO-d₆) δ (ppm): 3.43 (4H, bs, Piperazine-H), 3.80 (4H, bs, Piperazine-H), 4.27-4.28 (2H, d, CH₂), 5.34 (2H, s,CH₂), 6.67-6.68 (1H, d, Ar—H), 7.33-7.35 (2H, d, Ar—H), 7.40-7.42 (2H, d, Ar—H), 7.76-7.77 (1H, d, Ar—H), 7.81-7.82 (1H, d, Ar—H), 8.12-8.15 (1H, t, D₂O exchangeable) 8.38-8.39 (2H, d, Ar—H); M⁺ + 1 at 439.1; m.p: 174.6-176.5° C. |
| 69 | 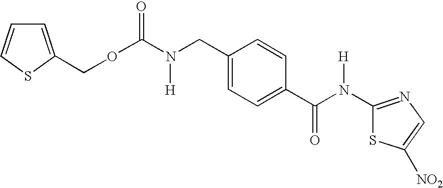 | ¹H NMR (DMSO-d₆) δ (ppm): 4.30- 4.31 (2H, d, CH₂), 5.22 (2H, s, CH₂), 7.01-7.03 (1H, t, Ar—H), 7.15-7.16 (1H, d, Ar—H), 7.43-7.45 (2H, d, Ar—H), 7.54-7.55 (1H, d, Ar—H), 7.93-7.95 (1H, t, D₂O exchangeable), 8.08-8.10 (2H, d, Ar—H), 8.72 (1H, s, Ar—H), 13.56 (1H, bs, D₂O exchangeable); M⁺ + 1 at 419.0; m.p: 173.3-174.3° C. |
| 70 | 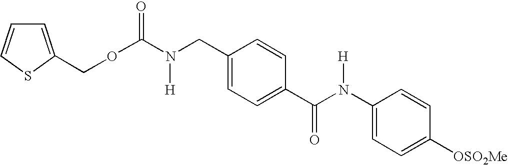 | ¹H NMR (DMSO-d₆) δ (ppm): 3.35 (3H, s, CH₃), 4.28-4.29 (2H, d, CH₂), 5.22 (2H, s, CH₂), 7.00-7.03 (1H, t, Ar—H), 7.14-7.15 (1H, d, Ar—H), 7.33-7.40 (4H, dd, Ar—H), 7.54-7.55 (1H, d, Ar—H), 7.84-7.85 (1H, t, D₂O exchangeable) 7.86-7.92 (4H, dd, Ar—H), 10.36 (1H, s, D₂O exchangeable); M⁺ + 1 at 461.0; m.p: 231.4-232.2° C. |
| 71 | 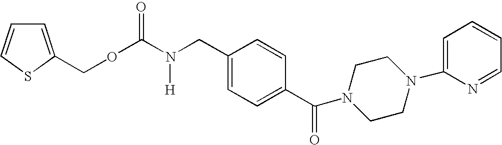 | ¹H NMR (CDCl₃) δ (ppm): 3.49 (4H, bs, Piperazine-H), 3.83 (4H, bs, Piperazine-H), 4.41-4.43 (2H, d, CH₂), 5.20-5.22 (1H, bs, D₂O exchangeable), 5.30 (2H, s, CH₂), 6.53-6.56 (1H, t, Ar—H), 6.98-7.00 (1H, q, Ar—H), 7.10 (1H, d, Ar—H), 7.32-7.34 (3H, t, Ar—H), 7.39-7.41 (2H, d, Ar—H), 8.32-8.33 (2H, d, Ar—H); M⁺ + 1 at 437.9; m.p: 129.3-130.3° C. |
| 72 | 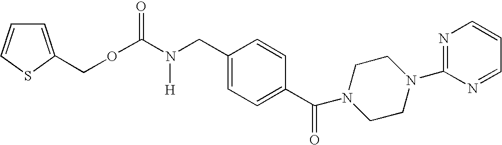 | ¹H NMR (CDCl₃) δ (ppm): 3.57 (4H, bs, Piperazine-H), 3.88 (4H, bs, Piperazine-H), 4.42-4.43 (2H, d, CH₂), 5.10 (2H, s, CH2), 5.30 (2H, s, CH₂), 6.65-6.69 (2H, q, Ar—H), 6.98-7.00 (1H, t, Ar—H), 7.10 (1H, s, Ar—H), 7.32-7.35 (2H, t, Ar—H), 7.40-7.42 (2H, d, Ar—H) 7.50-7.53 (1H, t, Ar—H), 8.19-8.20 (1H, d, Ar—H); M⁺ + 1 at 436.9; m.p: 143.6-144.6° C. |
| 73 | 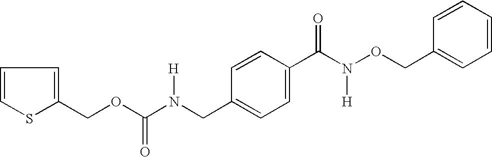 | ¹H NMR (DMSO-d₆) δ (ppm): 4.23- 4.24 (2H, d, CH₂), 4.91 (2H, s, CH₂), 5.20 (2H, s, CH₂) 6.99-7.00 (1H, d, Ar—H), 7.12-7.13 (1H, d, Ar—H), 7.30-7.36 (2H, d, Ar—H), 7.38-7.40 (3H, d, Ar—H), 7.43-7.44 (2H, d, Ar—H), 7.50-7.51 (1H, d, Ar—H), 7.65-7.67 (2H, d, Ar—H) 7.86-7.87 (1H, t, D₂O exchangeable), 11.73 (1H, s, D₂O exchangeable); M⁺ + 1 at 397.1; m.p: 180.9- 181.7° C. |
| 74 | 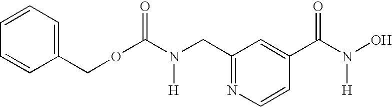 | ¹H NMR (DMSO-d₆) δ (ppm): 5.17 (2H, s, CH₂), 7.34-7.42 (6H, m, Ar—H), 8.15-8.21 (2H, d, Ar—H), 9.01-9.32 (1H, bs, D₂O exchangeable), 10.16 (1H, s, D₂O exchangeable); M⁺ + 1 at 387.9; m.p: 278.0-280.6° C. |
| 75 | 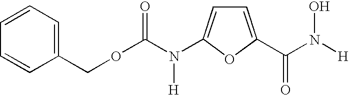 | ¹H NMR (DMSO-d₆) δ (ppm): 3.73 (2H, s, CH₂), 5.12-5.14 (2H, d, Ar—H), 6.09-6.12 (1H, d, Ar—H), 7.25-7.26 (1H, d, Ar—H), 7.32-7.40 (4H, m, Ar—H), 11.19 (1H, bs, D₂O exchangeable); M⁺ + 1 at 276.2; m.p: 310.1° C. (with decomposition) |
| 76 | 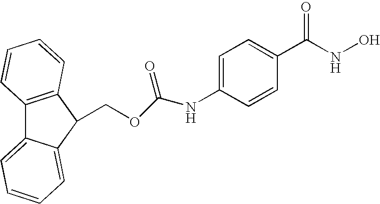 | ¹H NMR (DMSO-d₆) δ (ppm): 4.30- 4.34 (1H, t, CH), 4.51-4.53 (2H, d, CH₂), 7.34-7.37 (2H, t, Ar—H), 7.41-7.45 (2H, t, Ar—H), 7.49 (2H, d, CH₂), 7.66-7.68 (2H, d, Ar—H), 7.74-7.76 (2H, d, Ar—H), 7.91-7.93 (2H, d, Ar—H) 8.94 (1H, s, D₂O exchangeable), 9.96 (1H, s, D₂O exchangeable), 11.08 (1H, s, D₂O exchangeable); M⁺ + 1 at 375.0; m.p: 224.5-226.6° C. |

### Example: 77

### Synthesis of 3-thienylmethyl(4-{2-[acetyl(hydroxy)amino]-2-oxoethyl}-1,3-thiazol-2-yl)carbamate

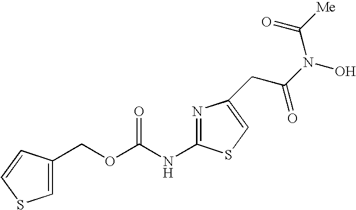

To a solution of 3-thienylmethyl {4-[2-(hydroxyamino)-2-oxoethyl]-1,3-thiazol-2-yl}carbamate (0.1 g, 0.32 mmol, prepared following procedure as described for example 1) in dichloromethane (4 mL), was added pyridine (0.07 mL, 0.96 mmol) and cooled to 0° C. followed by the dropwise addition of acetyl chloride (0.1 mL, 0.96 mmol) and the reaction mixture was stirred for 1 hour. The reaction mixture was diluted with dichloromethane (30 mL) and washed with water, the organic layer was dried on anhydrous Na₂SO₄, concentrated and triturated with dichloromethane/hexanes (1:1) (20 mL) to afford 0.04 g (35% yield) of the title compound as a colorless solid with m.p: 173-175° C. ¹H NMR (400 MHz, DMSO-d₆) δ (ppm): 2.15 (3H, s, —CH₃), 3.48 (2H, s, —CH₂), 5.20 (2H, s, —OCH₂), 6.92 (1H, s, Ar—H), 7.15-7.16 (1H, d, Ar—H) 7.56-7.58 (2H, t, Ar—H), 11.81 (2H, s, D₂O exchangeable). MS m/z: 355 (M⁺).

### Following Compounds are Prepared Following the Procedure Given in Example: 77

| | | |
|---|---|---|
| Exp. | Structure | Analytical data |

| | | |
|---|---|---|
| 78 | 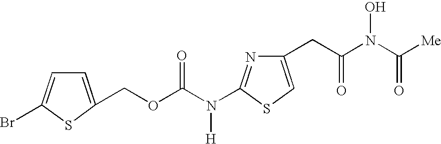 | ¹H NMR (DMSO-d₆) δ (ppm): 2.14 (3H, s, —CH₃), 3.40 (2H, s, —CH₂), 5.24 (2H, s, —OCH₂), 6.94 (1H, s, Ar—H), 7.08-7.09 (1H, s, Ar—H) 7.15-7.16 (1H, s, Ar—H), 11.75 (2H, s, D₂O exchangeable); M⁺ + 1 at 435; m.p: 163- 168° C. |
| 79 | 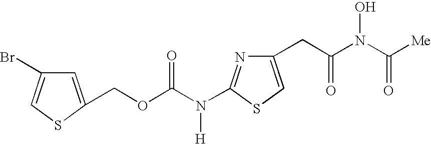 | ¹H NMR (DMSO-d₆) δ (ppm): 2.14 (3H, s, —CH₃), 3.37 (2H, s, —CH₂), 5.36 (2H, s, —OCH₂), 6.94 (1H, s, Ar—H), 7.25 (1H, s, Ar—H), 7.71 (1H, s, Ar—H), 11.84-11.90 (2H, d, D₂O exchangeable); M⁺ − 1 at 433; m.p: 184- 188° C. |
| 80 | 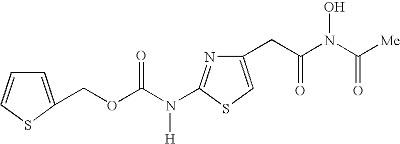 | ¹H NMR (DMSO-d₆) δ (ppm): 2.14 (3H, s, —CH₃), 3.48 (2H, s, —CH₂), 5.38 (2H, s, —OCH₂), 6.93 (1H, s, Ar—H), 7.03-7.05 (1H, m, Ar—H), 7.22 (1H, s, Ar—H), 7.58-7.59 (1H, d, Ar—H), 11.83 (2H, s, D₂O exchangeable); M⁺ + 1 at 355; m.p: 168-170° C. |
| 81 | 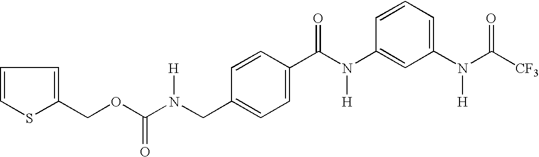 | ¹H NMR (DMSO-d₆) δ (ppm): 4.27-4.29 (2H, d, CH₂), 5.21 (2H, s, CH₂), 7.01-7.03 (1H, t, Ar—H), 7.15-7.16 (1H, d, Ar—H), 7.37- 7.40 (4H, t, Ar—H), 7.54-7.55 (1H, d, Ar—H), 7.58-7.60 (1H, t, Ar—H), 7.89-7.91 (2H, d, Ar—H), 7.92-7.93 (1H, t, D₂O exchangeable), 8.25 (1H, s, Ar—H), 10.33 (1H, s, D₂O exchangeable), 11.32 (1H, s, D₂O exchangeable); M⁺ + 1 at 478.0; m.p: 220.5-221.2° C. |
| 82 | 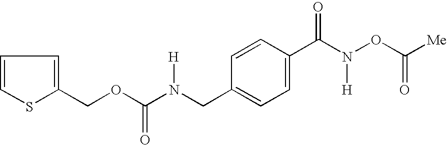 | ¹H NMR (DMSO-d₆) δ (ppm): 2.22 (3H, s, CH₃), 4.25-4.27 (2H, d, CH₂), 5.20 (2H, s, CH₂), 7.00-7.02 (1H, t, Ar—H), 7.14-7.15 (1H, d, Ar—H), 7.36-7.38 (2H, d, Ar—H), 7.53-7.54 (1H, d, Ar—H), 7.75-7.77 (2H, d, Ar—H), 7.88-7.90 (1H, t, D₂O exchangeable), 12.27 (1H, s, D₂O exchangeable); M⁺ + 1 at 349.0; m.p: 158.0-159.2° C. |
| 83 | 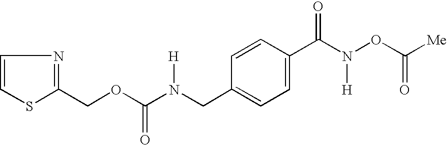 | ¹H NMR (DMSO-d₆) δ (ppm): 2.22 (3H, s, OCH₃), 4.28-4.29 (2H, d, CH₂), 5.32 (2H, s, CH₂), 7.38-7.40 (2H, d, Ar—H), 7.75-7.80 (4H, t, Ar—H), 8.12-8.13 (1H, t, D₂O exchangeable), 12.27 (1H, s, D₂O exchangeable); M⁺ + 1 at 350.1; m.p: 157.2- 158.8° C. |
| 84 | 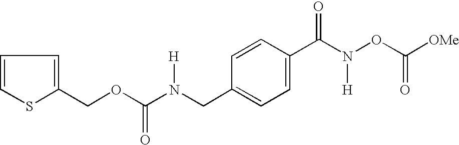 | ¹H NMR (DMSO-d₆) δ (ppm): 3.87 (3H, s, OCH₃), 4.26-4.27 (2H, d, CH₂), 5.20 (2H, s, CH₂), 7.00-7.02 (1H, t, Ar—H), 7.14-7.15 (1H, d, Ar—H), 7.37-7.39 (2H, d, Ar—H), 7.53- 7.55 (1H, d, Ar—H), 7.74-7.76 (2H, d, Ar—H), 7.90-7.93 (1H, t, D₂O exchangeable), 12.67 (1H, s, D₂O exchangeable); M⁺ + 1 at 365.0; m.p: 120.3-121.8° C. |
| 85 | 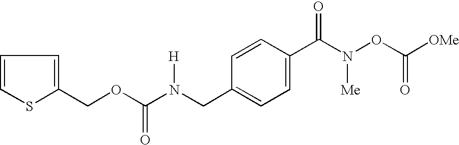 | ¹H NMR (DMSO-d₆) δ (ppm): 3.35 (3H, s, NCH₃), 3.79 (3H, s, OCH₃) 4.24-4.26 (2H, d, CH₂), 5.21 (2H, s, CH₂), 7.00-7.01 (1H, d, Ar—H), 7.14 (1H, s, Ar—H), 7.32-7.34 (2H, d, Ar—H), 7.48-7.50 (2H, d, Ar—H), 7.53-7.54 (1H, d, Ar—H), 7.89-7.90 (1H, t, D₂O exchangeable); M⁺ + 1 at 379.0. |
| 86 | 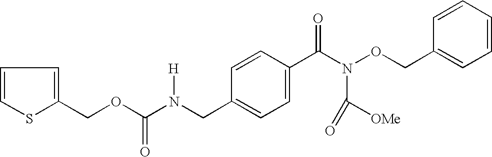 | ¹H NMR (DMSO-d₆) δ (ppm): 3.83 (3H, s, OCH₃), 4.22-4.24 (2H, d, CH₂), 5.18 (2H, s, CH₂), 5.19 (2H, s, CH₂), 7.00-7.02 (1H, t, Ar—H), 7.13-7.14 (1H, d, Ar—H), 7.32- 7.34 (2H, t, Ar—H), 7.35-7.37 (1H, d, Ar—H), 7.38-7.39 (4H, d, Ar—H) 7.53-7.54 (1H, d, D₂O exchangeable), 7.62-7.64 (2H, d, Ar—H), 7.88 (1H, t, D₂O exchangeable); M⁺ + 1 at 455.0; m.p: 91.5-92.0° C. |

### Example: 87

### Synthesis of pyridin-3-ylmethyl[4-(2-{[6-(hydroxyamino)-6-oxohexyl]amino}-2-oxoethyl)-1,3-thiazol-2-yl]carbamate

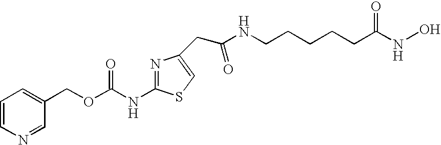

### Step: 1

### Preparation of methyl 6-{[(2-{[(pyridin-3-ylmethoxy)carbonyl]amino}-1,3-thiazol-4-yl)acetyl]amino}hexanoate

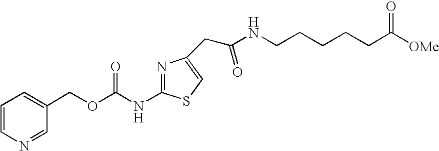

To a solution of (2-{[(pyridin-3-ylmethoxy)carbonyl]amino}-1,3-thiazol-4-yl)acetic acid (0.293 g, 1 mmol) (prepared following the procedure described in step 1 of example 2) in DMF (5 mL) was added DIPEA (0.4 mL, 2.4 mmol), EDCI (0.306 g, 1.6 mmol), and HOBt (0.042 g, 0.32 mmol) followed by methyl 6-aminohexanoate (0.116 g, 0.8 mmol). The reaction was stirred at room temperature for 12 hours, DMF was removed under reduced pressure and the crude material was taken up in EtOAc (50 mL) and washed with water. The organic layer was dried on anhydrous Na₂SO₄, concentrated and purified by silica gel column chromatography, using dichloromethane/MeOH (9.8:0.2) as the eluent to afford 0.260 g (62% yield) of the title compound as an off-white solid.

### Step: 2

### Preparation of pyridin-3-ylmethyl[4-(2-{[6-(hydroxyamino)-6-oxohexyl]amino}-2-oxoethyl)-1,3-thiazol-2-yl]carbamate

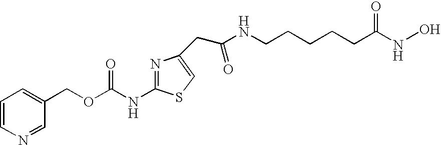

Hydroxylamine hydrochloride (0.625 g, 9 mmol) in methanol (2 ml) was mixed with KOH (0.5 g, 9 mmol) in methanol (3 ml) at 40° C., and cooled to 0° C., when a white precipitate was formed which was filtered. The filtrate was immediately added to the methyl 6-{[(2-{[(pyridin-3-ylmethoxy)carbonyl]amino}-1,3-thiazol-4-yl)acetyl]amino}hexanoate (0.21 g, 0.5 mmol) followed by the addition of KOH (0.042 g, 0.75 mmol), and the mixture was stirred at room temperature, for 1 hour. Around 20 mL of water was added and neutralized to a pH of 7 by dilute AcOH. On standing a colorless precipitate was forming which was filtered, dried and triturated with dichloromethane/hexanes (1:1) (20 mL), to afford the pure compound as a colorless solid (0.041 g, 20%) with m.p: 183-186° C. ¹H NMR (DMSO-d₆) δ (ppm): 1.21-1.22 (2H, d, —CH₂), 1.36-1.49 (4H, m, —CH₂), 1.92-1.96 (2H, d, —CH₂), 3.01-3.05 (2H, d, —CH₂), 3.41 (2H, s, —CH₂), 5.27 (2H, s, —OCH₂), 6.86 (1H, s, Ar—H), 7.44-7.47 (1H, m, Ar—H), 7.85-7.87 (1H, d, Ar—H), 7.97 (1H, s, Ar—H), 8.55-8.56 (1H, d, Ar—H), 8.63 (1H, s, D₂O exchangeable), 9.85 (1H, s, D₂O exchangeable), 10.34 (1H, s, D₂O exchangeable), 11.84 (1H, s, D₂O exchangeable). MS m/z: 422 (M+1).

### Following Compounds are Prepared Following the Procedure Given in Example: 87

| Exp. | Structure | Analytical data |
|---|---|---|
| 88 | 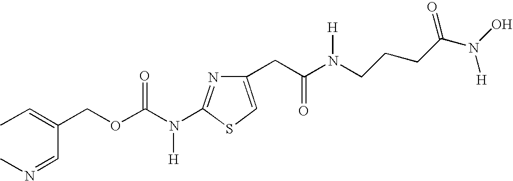 | ¹H NMR (DMSO-d₆) δ (ppm): 0.79-0.83 (2H, t, —CH₂), 1.48-1.63 (2H, m, —CH₂), 3.48 (2H, s, —CH₂), 3.79 (2H, s, —CH₂), 5.26 (2H, s, —OCH₂), 6.86 (1H, s, Ar—H), 7.44-7.47 (1H, m, Ar—H), 7.85-7.87 (1H, d, Ar—H), 8.12-8.14 (1H, d, D₂O exchangeable) 8.54- 8.56 (1H, d, Ar—H), 8.63 (1H, s, Ar—H), 9.53 (1H, s, D₂O exchangeable), 10.87 (2H, s, D₂O exchangeable); M⁺ + 1 at 394; m.p: 148-152° C. |
| 89 | 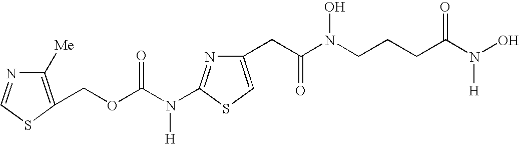 | ¹H NMR (DMSO-d₆) δ (ppm): 0.79-0.82 (2H, t, —CH₂), 1.48-1.62 (2H, m, —CH₂), 2.42 (3H, s, —CH₃), 3.47 (2H, s, —CH₂), 4.04-4.06 (2H, t, —CH₂), 5.39 (2H, s, —OCH₂), 6.57 (1H, s, Ar—H), 8.11-8.13 (1H, d, D₂O exchangeable), 8.84 (1H, s, D₂O exchangeable), 8.99 (1H, s, Ar—H), 10.43 (1H, s, D₂O exchangeable), 11.80 (1H, s, D₂O exchangeable); M⁺ + 1 at 414; m.p: 164-168° C. |
| 90 | 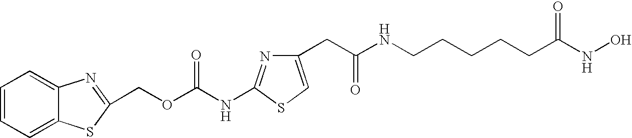 | ¹H NMR (DMSO-d₆) δ (ppm): 1.21-1.23 (2H, d, —CH₂), 1.36-1.40 (2H, t, —CH₂), 1.45-1.49 (2H, t, —CH₂), 1.91-1.94 (2H, t, —CH₂), 3.00-3.02 (2H, d, —CH₂), 3.39-3.42 (2H, d, CH₂), 5.64 (2H, s, —OCH₂), 6.88 (1H, s, Ar—H), 7.44-7.49 (1H, t, Ar—H), 7.52-7.56 (1H, t, Ar—H), 7.92 (1H, s, Ar—H), 8.01-8.03 (1H, d, Ar—H), 8.12-8.14 (1H, d, D₂O exchangeable), 8.67 (1H, s, D₂O exchangeable), 10.34 (1H, s, D₂O exchangeable), 12.15 (1H, s, D₂O exchangeable); M⁺ + 1 at 478; m.p: 161-165° C. |
| 91 | 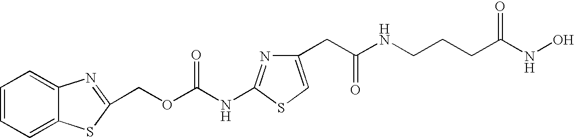 | ¹H NMR (DMSO-d₆) δ (ppm): 0.79-0.83 (2H, t, —CH₂) 1.23 (2H, s, —CH₂), 1.53-1.61 (2H, m, —CH₂), 3.50 (2H, s, CH₂), 5.64 (2H, s, —OCH₂), 6.89 (1H, s, Ar—H), 7.45-7.49 (1H, t, Ar—H), 7.52-7.56 (1H, t, Ar—H), 8.01-8.03 (1H, d, D₂O exchangeable), 8.12- 8.14 (2H, d, Ar—H), 8.86 (1H, s, D₂O exchangeable), 10.66 (1H, s, D₂O exchangeable), 12.17 (1H, s, D₂O exchangeable). M⁺ + 1 at 450; m.p: 133- 138° C. |
| 92 | 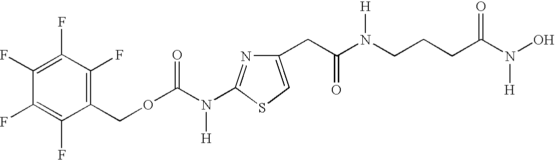 | ¹H NMR (DMSO-d₆) δ (ppm): 0.78-0.82 (2H, t, —CH₂), 1.46-1.62 (2H, m, —CH₂), 3.47 (2H, s, CH₂), 4.02-4.08 (2H, m, CH₂), 5.34 (2H, s, —OCH₂), 6.86-6.89 (1H, d, Ar—H), 8.10-8.12 (1H, d, D₂O exchangeable), 8.87 (2H, s, D₂O exchangeable), 10.67 (1H, s, D₂O exchangeable), 11.98 (1H, s, D₂O exchangeable); M⁺ + 1 at 483; m.p: 153- 158° C. |
| 93 | 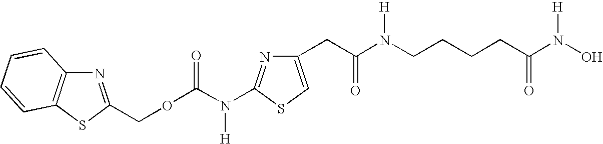 | ¹H NMR (DMSO-d₆) δ (ppm): 1.36-1.38 (2H, m, —CH₂), 1.46-1.48 (2H, m, —CH₂), 1.92-1.99 (2H, m, —CH₂), 3.02-3.03 (2H, d, —CH₂), 3.32 (2H, s, CH₂), 5.63 (2H, s, OCH₂), 6.86-6.88 (1H, d, Ar—H), 7.45-7.56 (2H, m, Ar—H), 8.01-8.03 (1H, d, Ar—H), 8.12-8.14 (1H, d, Ar—H), 8.85 (1H, s, D₂O exchangeable), 9.99 (1H, s, D₂O exchangeable), 10.58 (1H, s, D₂O exchangeable), 12.14 (1H, s, D₂O exchangeable); M⁺ + 1 at 464; m.p: 152-155° C. |
| 94 | 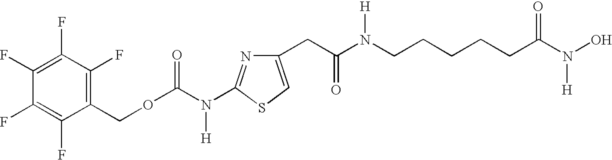 | ¹H NMR (DMSO-d₆) δ (ppm): 1.20-1.22 (2H, d, —CH₂), 1.35-1.37 (2H, d, —CH₂), 1.45-1.48 (2H, t, —CH₂), 1.90-1.93 (2H, t, —CH₂), 2.99-3.01 (2H, d, —CH₂), 3.33 (2H, s, —CH₂), 5.34 (2H, s, —OCH₂), 6.85 (1H, s, Ar—H), 7.90 (1H, t, Ar—H), 8.67 (1H, s, D₂O exchangeable), 10.33 (1H, s, D₂O exchangeable), 11.89 (1H, s, D₂O exchangeable); M⁺ + 1 at 511; m.p: 151-154° C. |
| 95 | 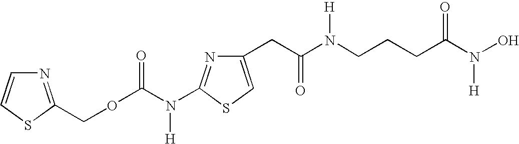 | ¹H NMR (DMSO-d₆) δ (ppm): 0.80-0.84 (2H, t, —CH₂), 1.50-1.56 (2H, m, —CH₂), 1.60-1.62 (2H, d, —CH₂), 3.50 (2H, s, —CH₂), 5.49 (2H, s, —OCH₂), 6.89 (1H, s, Ar—H), 7.77-7.78 (2H, d, Ar—H), 8.12-8.15 (1H, d, D₂O exchangeable), 8.87 (1H, s, D₂O exchangeable), 10.66 (1H, s, D₂O exchangeable), 12.02 (1H, s, D₂O exchangeable), M⁺ + 1 at 400; m.p: 157- 161° C. |
| 96 | 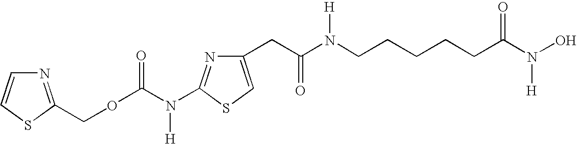 | ¹H NMR (DMSO-d₆) δ (ppm): 1.21-1.23 (2H, d, —CH₂), 1.36-1.40 (2H, t, —CH₂), 1.45-1.49 (2H, t, —CH₂), 1.91-1.94 (2H, t, —CH₂), 3.00-3.02 (2H, d,—CH₂), 3.40 (2H, s, CH2), 5.50 (2H, s, —OCH₂), 6.87 (1H, s, Ar—H), 7.80-7.84 (2H, d, Ar—H), 7.91 (1H, s, D₂O exchangeable), 8.67 (1H, s, D₂O exchangeable), 10.34 (1H, s, D₂O exchangeable), 12.00 (1H, s, D₂O exchangeable); M⁺ + 1 at 468; m.p: 158-162° C. |
| 97 | 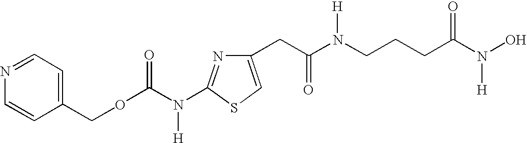 | ¹H NMR (DMSO-d₆) δ (ppm): 0.79-0.83 (2H, t, —CH₂), 1.40-1.75 (2H, m, —CH₂), 3.45-3.49 (2H, d, —CH₂), 3.98-4.02 (2H, d, —CH₂), 5.27 (2H, s, —OCH₂), 6.86 (1H, s, Ar—H), 7.37-7.38 (2H, d, Ar—H), 8.13 (1H, s, D₂O exchangeable), 8.57-8.58 (2H, d, Ar—H), 8.88 (1H, s, D₂O exchangeable), 10.67 (2H, s, D₂O exchangeable); M⁺ + 1 at 394; m.p: 168-172° C. |
| 98 | 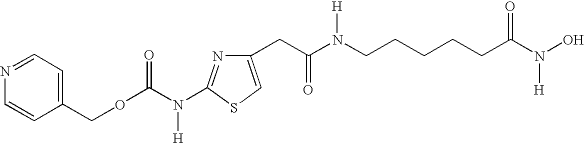 | ¹H NMR (DMSO-d₆) δ (ppm): 1.21-1.23 (2H, d, —CH₂), 1.36-1.49 (4H, m, —CH₂), 1.91-1.94 (2H, d, —CH₂), 3.01-3.02 (2H, d, —CH₂), 3.41 (2H, s, CH₂), 5.27 (2H, s, —OCH₂), 6.85, (1H, s, Ar—H), 7.37-7.38 (2H, d, Ar—H), 7.91 (1H, s, Ar—H), 8.57-8.58 (2H, d, Ar—H), 8.66 (1H, s, D₂O exchangeable), 9.99 (1H, s, D₂O exchangeable), 10.33 (1H, s, D₂O exchangeable), 11.95 (1H, s, D₂O exchangeable); M⁺ + 1 at 422; m.p: 174- 175.8° C. |
| 99 | 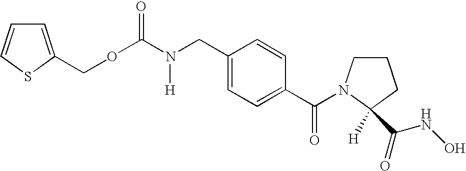 | ¹H NMR (DMSO-d₆) δ (ppm); 1.79-1.83 (2H, m, —CH₂), 1.89-2.13 (2H, m, CH₂), 3.40-3.48 (2H, t, CH₂), 4.23-4.25 (2H, d, CH₂), 4.31-4.32 (1H, t, CH), 5.21 (2H, s, —CH₂), 7.01-7.02 (1H, d, Ar—H), 7.14 (1H, s, Ar—H), 7.26- 7.31 (2H, t, Ar—H), 7.50- 7.55 (3H, q, Ar—H), 7.88- 7.90 (1H, t, D₂O exchangeable), 8.85 (1H, s, D₂O exchangeable), 10.64 (1H, d, D₂O exchangeable); M⁺ + 1 at 403.9; m.p: 90.6-91.5° C. |
| 100 | 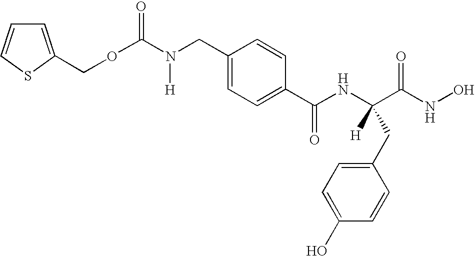 | ¹H NMR (DMSO-d₆) δ (ppm): 2.87-2.89 (2H, d, CH₂), 4.22-4.24 (2H, d, CH₂), 4.48-4.50 (1H, t, CH), 5.20 (2H, s, CH₂), 6.61-6.63 (2H, d, Ar—H), 7.00-7.02 (1H, t, Ar—H), 7.08-7.09 (2H, d, Ar—H), 7.14-7.15 (1H, d, Ar—H), 7.27-7.29 (2H, d, Ar—H) 7.53-7.54 (1H, d, Ar—H) 7.74-7.76 (2H, d, Ar—H), 7.85-7.87 (1H, t, D₂O exchangeable) 8.48-8.50 (1H, d, D₂O exchangeable) 8.86 (1H, s, D₂O exchangeable), 9.14 (1H, s, D₂O exchangeable), 10.74 (1H, s, D₂O exchangeable). M⁺ + 1 at 469.8; m.p: 162.6-163.4° C. |
| 101 | 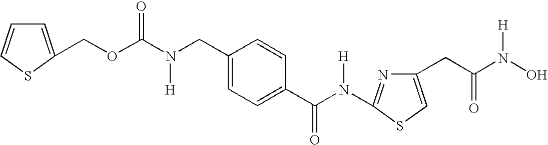 | ¹H NMR (DMSO-d₆) δ (ppm): 3.42 (2H, s, CH₂), 4.28-4.29 (2H, d, CH₂), 5.22 (2H, s, CH₂), 6.88 (1H, s, Ar—H), 7.02-7.04 (1H, d, Ar—H), 7.15 (1H, s, Ar—H), 7.39-7.41 (2H, d, Ar—H), 7.53-7.55 (1H, d, Ar—H), 7.93-7.94 (1H, t, Ar—H), 8.01-8.02 (1H, d, Ar—H), 8.03-8.04 (1H, s, D₂O exchangeable), 8.87 (1H, s, D₂O exchangeable), 10.61 (1H, s, D₂O exchangeable), 12.59 (1H, s, D₂O exchangeable); M⁺ + 1 at 447.0; m.p: 203.2- 204.0° C. |
| 102 | 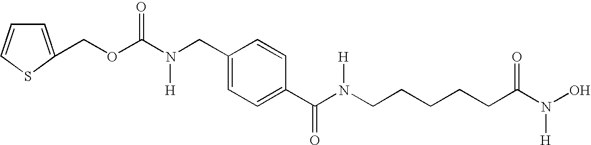 | ¹H NMR (DMSO-d₆) δ (ppm): 1.24-1.27 (2H, t, CH₂) 4.27-4.29 (2H, d, —CH₂), 5.47 (2H, s, —CH₂), 7.33-7.35 (2H, d, Ar—H), 7.44-7.48 (1H, t, Ar—H), 7.52-7.56 (1H, dd, Ar—H), 7.70-7.72 (2H, d, Ar—H), 7.99-8.01 (1H, d, Ar—H), 8.12-8.14 (1H, d, Ar—H), 8.21-8.24 (1H, t, D₂O exchangeable), 9.02 (1H, s, D₂O exchangeable), 11.19 (1H, s, D₂O exchangeable); M⁺ 1 at 420.1; m.p: 170.4- 172.1° C. |
| 103 | 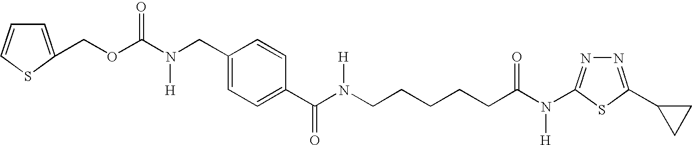 | ¹H NMR (DMSO-d₆) δ (ppm): 0.95-0.96 (2H, t, cyclo prop), 1.10-1.12 (2H, t, cyclo prop), 1.30-1.35 (2H, d, CH₂), 1.50-1.51 (2H, d, CH₂), 1.61 (2H, s, CH₂), 2.42-2.44 (1H, m, CH), 2.45-2.48 (2H, m, CH₂), 3.22-3.24 (2H, d, CH₂), 4.23-4.24 (2H, t, CH₂), 5.20 (2H, s, CH₂), 7.00-7.02 (1H, t, Ar—H), 7.14 (1H, s, Ar—H), 7.29-7.31 (2H, d, Ar—H), 7.53-7.54 (1H, d, Ar—H) 7.75-7.77 (2H, d, Ar—H), 7.88 (1H, s, D₂O exchangeable), 8.40 (1H, s, D₂O exchangeable), 12.35 (1H, s, D₂O exchangeable); M⁺ + 1 at 527.8; m.p: 178.6-180.6° C. |

### Example: 104

### Synthesis of pyridin-4-ylmethyl[4-(2-{[2-({[(4-cyanophenyl)amino]carbonyl}-amino)phenyl]amino}-2-oxoethyl)-1,3-thiazol-2-yl]carbamate

### Step: 1

### Preparation of (2-{[(pyridin-4-ylmethoxy)carbonyl]amino}-1,3-thiazol-4-yl)acetic acid

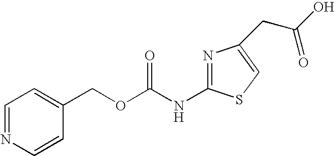

To a suspension of 1,1′-carbonylbis(1H-imidazole) (5.93 g, 36.6 mmol) in THF (27 mL) at 0-5° C. was added pyridine-4-methanol (4 g, 36.6 mmol) in THF (12 mL), and stirred at room temperature for 5 hours. The above reaction mixture was added to a suspension of 2-amino-4-thiazole acetic acid (5.8 g, 36.6 mmol), DBU (5.5 g, 36.6 mmol) and triethylamine (3.6 g, 36.6 mmol) in THF (50 mL) and stirred at room temperature overnight. The THF was removed under reduced pressure and the crude compound was taken up in dichloromethane (200 mL) and washed with water, the aqueous layer was acidified to a pH of 6 to afford a pale yellow colored precipitate which was filtered and dried to give the title compound (3.2 g, 30% yield).

### Step: 2

### Preparation of pyridin-4-ylmethyl(4-{2-[(2-aminophenyl)amino]-2-oxoethyl}-1,3-thiazol-2-yl)carbamate

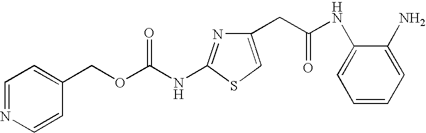

To a suspension of (2-{[(pyridin-4-ylmethoxy)carbonyl]amino}-1,3-thiazol-4-yl)acetic acid (0.293 g, 1 mmol) in DMF (5 mL) was added DIPEA (0.58 g, 4.5 mmol), EDCI (0.58 g, 3 mmol), HOBt (0.081 g, 0.6 mmol) followed by the o-phenylenediamine (0.12 g, 1.1 mmol). The reaction mixture was stirred at room temperature for 12 hours DMF was removed under reduced pressure and the crude material was taken up in EtOAc (50 mL) and washed with water, the organic layer was dried on anhydrous Na₂SO₄, concentrated and purified by dissolving in dichloromethane/methanol (3:1) (2 mL) and precipitating with hexanes. The precipitate was filtered to afford the required compound as a pure colorless solid (0.170 g, 44%) with m.p: 188-191° C.

### Step: 3

### Preparation of pyridin-4-ylmethyl[4-(2-{[2-({[(4-cyanophenyl)amino]-carbonyl}-amino)phenyl]amino}-2-oxoethyl)-1,3-thiazol-2-yl]carbamate

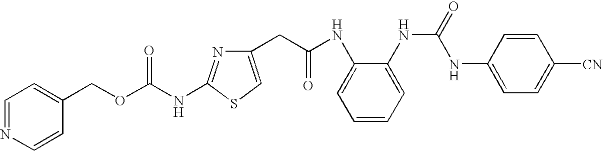

To a solution of pyridin-4-ylmethyl (4-{2-[(2-aminophenyl)amino]-2-oxoethyl}-1,3-thiazol-2-yl)carbamate (0.150 g, 3.9 mmol) in THF (5 mL) was added dropwise 4-cyanophenylisocyanate (0.056 g, 3.9 mmol) and stirred the reaction mixture at room temperature for 4 hours. The THF was removed under reduced pressure and the crude compound was purified by silicagel column chromatography, using dichloromethane/MeOH (9.8:0.2) as the eluent to afford 0.18 g (90% yield) of the title compound as a colorless solid with m.p.: 186-188° C. ¹H NMR (DMSO-d₆) δ (ppm): 3.75 (2H, s, CH₂), 5.26 (2H, s, —OCH₂), 7.00 (1H, s, Ar—H), 7.11-7.15 (1H, m, Ar—H), 7.19-7.23 (1H, m, Ar—H), 7.38-7.39 (3H, d, Ar—H), 7.60-7.71 (5H, m, Ar—H), 8.05 (1H, s, D₂O exchangeable), 8.57-8.58 (2H, d, Ar—H), 9.65-9.72 (2H, m, D₂O exchangeable), 11.95 (1H, s, D₂O exchangeable). MS m/z: 527 (M⁺).

### Following Compound are Prepared Following the Procedure Given in Example 104

| Exp. | Structure | Analytical data |
|---|---|---|
| 105 | 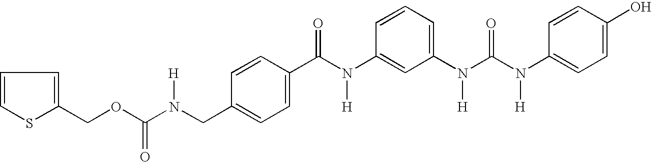 | ¹H NMR (DMSO-d₆) δ (ppm): 4.28-4.29 (2H, d, CH₂), 5.22 (2H, s, CH₂), 7.02-7.03 (1H, d, Ar—H), 7.15-7.16 (1H, d, Ar—H), 7.24- 7.26 (2H, d, Ar—H), 7.38-7.41 (3H, t, Ar—H), 7.54-7.55 (1H, d, Ar—H), 7.63-7.65 (2H, d, Ar—H), 7.72-7.75 (2H, d, Ar—H) 7.89- 7.90 (2H, d, D₂O exchangeable), 7.91-7.92 (1H, s, D₂O exchangeable), 7.99 (1H, s, Ar—H), ) 8.95 (1H, s, D₂O exchangeable), 9.16 (1H, s, D₂O exchangeable), 10.22 (1H, s, D₂O exchangeable); M⁺ + 1 at 526.1; m.p: 226.1-226.8° C. |

### Example: 106

### Synthesis of 2-thienylmethyl(4-[({(2-{1,3-benzodioxol-5-ylcarbonyl}amino)-phenyl}amino)carbonyl]benzyl)carbamate

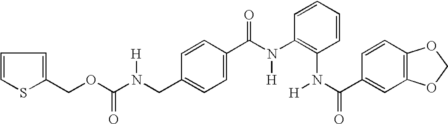

### Step: 1

### Preparation of 2-thienylmethyl(4-{[(2-aminophenyl)amino]carbonyl}benzyl)-carbamate

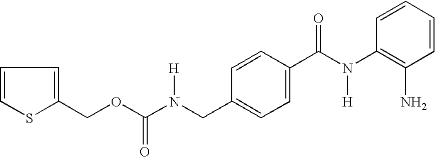

The above compound was prepared following the procedure described in example 2.

### Step: 2

### Preparation of 2-thienylmethyl(4-[({(2-{1,3-benzodioxol-5-ylcarbonyl}amino)-phenyl}amino)carbonyl]benzyl)carbamate

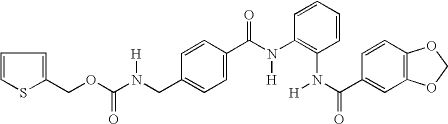

The coupling of 2-thienylmethyl (4-{[(2-aminophenyl)amino]carbonyl}-benzyl)carbamate with piperonylic acid, (was done following the same procedure as described in step 2, of example 2), afforded the title compound as a pale brown colored paste (30% yield). ¹H NMR (DMSO-d₆) δ (ppm): 4.24-4.30 (2H, m, CH₂), 5.21 (2H, s, CH₂), 6.12 (2H, s, CH₂), 7.04-7.06 (2H, d, Ar—H), 7.14-7.15 (1H, d, Ar—H), 7.27-7.29 (21-1, t, Ar—H), 7.37-7.39 (21-1, d, Ar—H), 7.47 (1H, s, Ar—H), 7.52-7.56 (2H, t, Ar—H), 7.65-7.68 (2H, t, Ar—H), 7.87-7.89 (2H, d, Ar—H), 7.89-7.90 (1H, t, D₂O exchangeable), 9.93 (1H, s, D₂O exchangeable), 10.00 (1H, s, D₂O exchangeable). MS m/z: 530.1 (M+1).

### Following Compounds are Prepared Following the Procedure Given in Example 106

| Exp. | Structure | Analytical data |
|---|---|---|
| 107 | 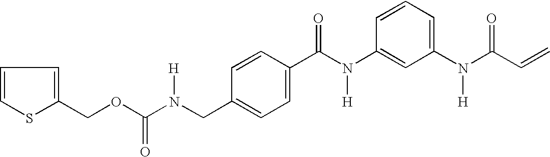 | ¹H NMR (DMSO-d₆) δ (ppm): 4.27- 4.28 (2H, d, CH₂), 5.22 (2H, s, CH₂), 5.74-5.77 (1H, d, Ar—H), 6.24-6.28 (1H, d, Ar—H), 6.44-6.46 (1H, d, Ar—H), 7.01-7.03 (1H, t, Ar—H), 7.15 (1H, s, Ar—H), 7.26-7.28 (1H, d, Ar—H), 7.30 (1H, s, D₂O exchangeable), 7.30-7.32 (3H, d, Ar—H), 7.89-7.90 (2H, d, Ar—H), 8.18 (1H, s, Ar—H), 10.18 (1H, t, HC═C<) 10.24 (1H, s, D₂O exchangeable); M⁺ + 1 at 436.0. |
| 108 | 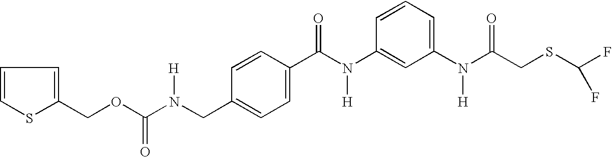 | ¹H NMR (DMSO-d₆) δ (ppm): 1.16 (1H, s, CH), 3.76 (2H, s, CH₂), 4.28- 4.29 (2H, d, CH₂), 5.22 (2H, s, CH₂), 7.02-7.04 (1H, t, Ar—H), 7.15- 7.16 (1H, d, Ar—H), 7.28-7.29 (1H, d, Ar—H), 7.33-7.35 (3H, t, Ar—H), 7.37-7.39 (1H, t, Ar—H) 7.53-7.54 (1H, d, Ar—H) 7.89-7.90 (1H, t, D₂O exchangeable), 7.91-7.92 (2H, t, Ar—H) 8.11 (1H, S, Ar—H) 10.25 (1H, s, D₂O exchangeable) 10.30 (1H, s, D₂O exchangeable); M⁺ + 1 at 506.0; m.p: 200.5-201.8° C. |

### Example: 109

### Synthesis of ethyl{methoxy[4-({[(2-thienylmethoxy)carbonyl]amino}-methyl)benzoyl]amino}acetate

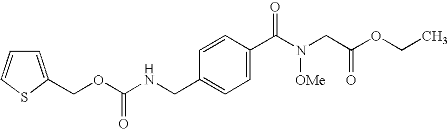

### Step: 1

### Preparation of 4-({[(2-thienylmethoxy)carbonyl]amino}methyl)benzoic acid

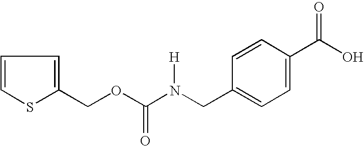

The above compound was synthesized by coupling of thiophene-2-methanol with 4-aminomethyl benzoic acid following the same procedure as described in step 1 of example 2.

### Step: 2

### Preparation of 2-thienylmethyl{4-[(methoxyamino)-carbonyl]benzyl}-carb-amate

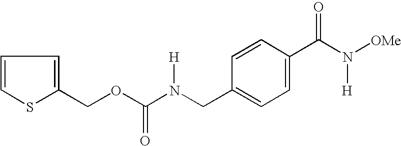

The coupling of 4-({[(2-thienylmethoxy)carbonyl]amino}methyl)benzoic acid with methoxylamine hydrochloride, was done following the same procedure as described in the step 2, of example 2.

### Step: 3

### Preparation of ethyl{methoxy[4-({[(2-thienylmethoxy)carbonyl]amino}-methyl)benzoyl]amino}acetate

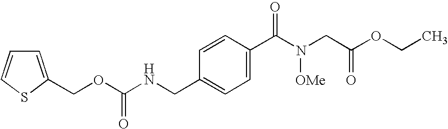

To a solution of 2-thienylmethyl {4-[(methoxyamino)carbonyl]benzyl}-carbamate (0.1 g, 0.3 mmol) in DMF (10 ml), ethyl bromo acetate (0.04 ml, 0.375 mmol) and potassium carbonate (0.13 g, 0.9 mmol) were added and stirred at room temperature, overnight. Subsequently the reaction mixture was diluted with ethyl acetate and washed with water. The organic layer was dried over anhydrous Na₂SO₄, concentrated and purified by silica-gel column chromatography using ethyl acetate/hexanes (20:80), to afford the title compound as sticky mass (100 mg, 78.7% yield). ¹H NMR (CDCl₃) δ (ppm): 1.29-1.32 (3H, t, CH₃), 3.58 (3H, s, OCH₃), 4.23-4.28 (2H, q, CH₂), 4.42-4.46 (4H, m, CH₂), 5.14 (1H, bs, D₂O exchangeable), 5.29 (2H, s, CH₂), 6.98-7.00 (1H, t, Ar—H), 7.10 (1H, s, Ar—H), 7.31-7.33 (3H, d, Ar—H), 7.70-7.72 (2H, d, Ar—H). MS m/z: 407.1 (M+1).

### Example: 110

### Synthesis of 2-thienylmethyl{4-[(E)-amino(hydroxyimino)-methyl]-phenyl}-carbamate

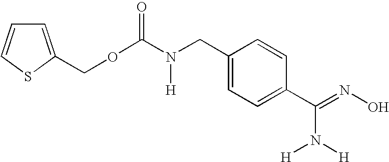

### Step: 1

### Synthesis of 2-thienylmethyl(4-cyanophenyl)carbamate

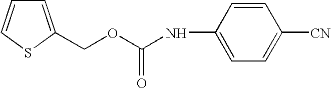

To a solution of thiophene-2-methanol (1 g, 8.8 mmol) in dichloromethane (10 mL), 4-cyanophenyl isocyanate (1.4 g, 6.0 mmol), triethylamine (2.4 mL, 17.5 mmol) were added and stirred at room temperature for 6 hours. The reaction mixture was diluted with ethyl acetate and washed with water. The organic layer was dried over anhydrous Na₂SO₄ and concentrated to afford the title compound as a pale yellow colored solid (1.7 g, 77.27% yield).

### Step: 2

### Synthesis of 2-thienylmethyl{4-[(E)-amino(hydroxyimino)methyl]phenyl}-carbamate

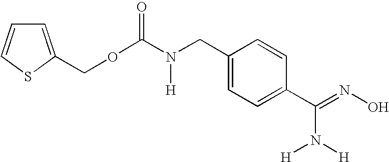

To a solution of 2-thienylmethyl (4-cyanophenyl)carbamate (1 g, 3.87 mmol) in ethanol (20 mL) was added hydroxylamine hydrochloride (0.54 g, 7.75 mmol), followed by sodium carbonate (0.82 g, 7.75 mmol) and refluxed for 4 hours. The solid that appeared was filtered and dried to afford the title compound as colorless solid (0.8 g, 71.4% yield) with m.p.: 192.8-194° C. ¹H NMR (DMSO-d₆) δ (ppm): 5.32 (2H, s, CH₂), 5.72 (21-1, bs, D₂O exchangeable), 7.03-7.05 (1H, t, Ar—H), 7.21 (1H, s, Ar—H), 7.43-7.45 (2H, d, Ar—H), 7.57-7.59 (3H, t, Ar—H), 9.50 (1H, s, D₂O exchangeable), 9.86 (1H, s, D₂O exchangeable). MS m/z: 291.9 (M+1).

### Anti-Cancer Experimental Methods

### Anti-Cancer Screen:

Experimental drugs are screened for anti-cancer activity in three cell lines for their GI₅₀, TGI and LC₅₀ values (using five concentrations for each compound). The cell lines are maintained in DMEM containing 10% fetal bovine serum. 96 well micro titer plates are inoculated with cells in 100 □L for 24 hours at 37° C., 5% CO₂, 95% air and 100% relative humidity. 5000 HCT 116 cells/well, 5000 NCIH 460 cells/well and 5000 U251 cells/well are plated. A separate plate with these cell lines is also inoculated to determine cell viability before the addition of the compounds (T₀).

### Addition of Experimental Drugs:

Following 24-hour incubation, experimental drugs are added to the 96 well plates. Each plate contains one of the above cell lines and the following in triplicate: five different concentrations (0.01, 0.1, 1, 10 and 100 □M) of four different compounds, appropriate dilutions of a cytotoxic standard and control (untreated) wells. Compounds are dissolved in DMSO to make 20 mM stock solutions on the day of drug addition and frozen at −20° C. Serial dilutions of these 20 mM stock solutions are made in complete growth medium such that 100 □L of these drug solutions in medium, of final concentrations equaling 0.01, 0.1, 1, 10 and 100 □M can be added to the cells in triplicate. Standard drugs whose anti-cancer activity has been well documented and which are regularly used are doxorubicin and SAHA.

### End-Point Measurement:

For T₀ measurement, 24 hours after seeding the cells, 10 □L of 3-(4,5-Dimethyl-2-thiazolyl)-2,5-diphenyl-2H-tetrazolium (MTT) solution per well is added and incubation carried out for 3 hours at 37° C., 5% CO₂, 95% air and 100% relative humidity, protected from light. Cells incubated with compounds for 48 hours are treated similarly except with the addition of 20 □l MTT solution per well and a subsequent incubation under the same conditions. After 3 hours of MTT incubation, well contents are aspirated carefully followed by addition of 150 □L DMSO per well. Plates are agitated to ensure solution of the formazan crystals in DMSO and absorbance read at 570 nm.

### Calculation of GI₅₀, TGI LC₅₀:

Percent growth is calculated for each compound's concentration relative to the control and zero measurement wells (T₀; viability right before compound addition). If a test well's O.D. value is greater than the T₀ measurement for that cell line

%Growth=(test−zero)/(control−zero)×100

If a test well's O.D. value is lower than the T₀ measurement for that cell line, then,

%Growth=(test−zero)/zero×100

Plotting % growth versus experimental drug concentration, GI₅₀ is the concentration required to decrease % growth by 50%; TGI is the concentration required to decrease % growth by 100% and LC₅₀ is the concentration required to decrease % growth by 150%.

Anticancer and HDAC inhibition Activity

| | NCIH460 | | | HCT116 | | | MDAMB-231/U 251 | | | Mean | HDAC Inhibition | | |
|---|---|---|---|---|---|---|---|---|---|---|---|---|---|
| S. No | GI₅₀ | TGI | LC₅₀ | GI₅₀ | TGI | LC₅₀ | GI₅₀ | TGI | LC₅₀ | GI₅₀ | 1 μM | 10 μM | IC₅₀ (μM) |
| 1 | 0.01 | 0.5 | >100 | 0.25 | 4 | >100 | 1 | >100 | >100 | 0.4 | 1.3 | 31.5 | |
| 2 | >100 | >100 | >100 | >100 | >100 | >100 | >100 | >100 | >100 | >100 | 0.8 | 22.42 | |
| 3 | 30 | >100 | >100 | >100 | >100 | >100 | 32 | >100 | >100 | 31 | −4.03 | 22.89 | |
| 4 | 20 | >100 | >100 | >100 | >100 | >100 | 6 | >100 | >100 | 13 | 5.6 | 38.13 | |
| 5 | 18 | 76 | >100 | 40 | 80 | >100 | 6 | 78 | >100 | 21.3 | 0.65 | 29.29 | |
| 6 | 2 | 80 | >100 | 65 | >100 | >100 | 5 | 82 | >100 | 24 | 8.8 | 47.27 | |
| 7 | 10 | 70 | >100 | 25 | 80 | >100 | 15 | 74 | >100 | 16.7 | 7.07 | 46.41 | |
| 8 | >100 | >100 | >100 | >100 | >100 | >100 | 0.5 | >100 | >100 | >100 | 0.96 | 17.80 | |
| 9 | 73 | >100 | >100 | 48 | >100 | >100 | 38 | >100 | >100 | 53 | 17.52 | 46.21 | |
| 10 | 55 | >100 | >100 | >100 | >100 | >100 | 0.4 | 27 | >100 | 26.7 | −9.85 | 0.14 | |
| 11 | 7 | >100 | >100 | >100 | >100 | >100 | 5 | 10 | >100 | 6 | NA | NA | |
| 12 | 50 | 100 | >100 | >100 | >100 | >100 | 50 | >100 | >100 | 50 | NA | 10.83 | |
| 13 | 2.8 | 25 | >100 | >100 | >100 | >100 | 0.09 | >100 | >100 | >100 | | | |
| 14 | 10 | >100 | >100 | 80 | >100 | >100 | >100 | >100 | >100 | 45 | NA | NA | |
| 15 | 60 | >100 | >100 | 100 | >100 | >100 | 60 | >100 | >100 | 73.3 | NA | NA | |
| 16 | 8 | >100 | >100 | >100 | >100 | >100 | 20 | >100 | >100 | 14 | NA | NA | |
| 17 | 80 | >100 | >100 | 82 | >100 | >100 | 2.5 | 50 | >100 | 54.8 | NA | 3.61 | |
| 18 | 100 | >100 | >100 | >100 | >100 | >100 | 12 | >100 | >100 | >100 | 2.19 | 7.99 | |
| 19 | 1 | 60 | >100 | 34 | >100 | >100 | 7.6 | 80 | >100 | 14.2 | NA | 0.50 | |
| 20 | 18 | 96 | >100 | 31 | >100 | >100 | 42 | >100 | >100 | 30.33 | NA | NA | |
| 21 | 15 | >100 | >100 | 8 | >100 | >100 | 4 | 9 | >100 | 9 | 4.41 | 0.57 | |
| 22 | 48 | >100 | >100 | 53 | >100 | >100 | 10 | >100 | >100 | 37 | −1.23 | 9.42 | |
| 23 | >100 | >100 | >100 | >100 | >100 | >100 | 31 | >100 | >100 | >100 | 11.34 | 5.18 | |
| 24 | >100 | >100 | >100 | >100 | >100 | >100 | >100 | >100 | >100 | >100 | 1.34 | 3.17 | |
| 25 | 92 | >100 | >100 | 65 | >100 | >100 | 85 | >100 | >100 | 80.67 | −0.29 | 6.62 | |
| 26 | NA | NA | NA | NA | NA | NA | NA | NA | NA | NA | NA | NA | |
| 27 | 34 | 64 | 100 | 7 | 21 | 72 | >100 | >100 | >100 | 24.9 | 67.65 | 81.85 | 0.58 |
| 28 | 30 | 51 | >100 | 5 | 15 | 62 | 5 | 30 | 70 | 13 | 50.8 | 63.3 | 0.123 |
| 29 | 0.4 | 22.5 | 82.5 | 0.98 | 3.5 | 48 | 3.1 | >100 | >100 | 1.5 | 72.7 | 85 | 0.021 |
| 30 | 0.02 | 0.05 | 73 | 0.23 | 7 | 61 | 0.78 | >100 | >100 | 0.3 | 84 | 84.6 | 0.14 |
| 31 | 0.02 | 0.9 | 92 | 0.02 | 0.97 | 39 | 32 | >100 | >100 | 10.7 | 82.6 | 87.4 | 0.13 |
| 32 | 5 | 42 | 95 | 5 | 25 | >100 | 11 | 45 | 80 | 7 | 78.12 | 92.67 | |
| 33 | 1 | 68 | >100 | 4.5 | 20 | >100 | 3 | 8 | 60 | 2.8 | 82.77 | 92.11 | 0.25 |
| 34 | 58 | >100 | >100 | 60 | >100 | >100 | 30 | >100 | >100 | 49.3 | 62.1 | 98.6 | |
| 35 | 20 | 50 | 80 | 5 | 12 | 60 | 0.05 | 15 | 70 | 8.4 | 74.16 | 85.41 | |
| 36 | 68 | >100 | >100 | 35 | 60 | 85 | 5.5 | 10 | 85 | 36.2 | 50.51 | 78.58 | |
| 37 | 30 | 58 | 90 | 6 | 20 | 65 | 4 | 18 | 95 | 13.3 | 67.04 | 85.01 | |
| 38 | 22 | 52 | 82 | 5 | 20 | 65 | 0.5 | 8 | 68 | 9.2 | 21.46 | 96.53 | |
| 39 | 16 | 54 | 82 | 5.5 | 10 | 62 | 0.4 | 5 | 65 | 7.3 | 82.75 | 100 | |
| 40 | >100 | >100 | >100 | 100 | >100 | >100 | 14 | >100 | >100 | >100 | −1.9 | 8.6 | |
| 41 | 6 | >100 | >100 | 58 | >100 | >100 | 9 | >100 | >100 | 24.3 | NA | NA | |
| 42 | >100 | >100 | >100 | >100 | >100 | >100 | 0.7 | >100 | >100 | >100 | NA | NA | |
| 43 | >100 | >100 | >100 | >100 | >100 | >100 | >100 | >100 | >100 | >100 | 1.9 | 8.10 | |
| 44 | 42 | >100 | >100 | 21 | >100 | >100 | 5 | 9 | >100 | 22.67 | −0.16 | −0.74 | |
| 45 | 90 | >100 | >100 | >100 | >100 | >100 | 2.5 | 65 | >100 | >100 | 4.28 | −0.76 | |
| 46 | 70 | >100 | >100 | 88 | >100 | >100 | 40 | 90 | >100 | 66 | −4.01 | −2.70 | |
| 47 | 56 | >100 | >100 | 52 | >100 | >100 | 37 | 74 | >100 | 48.33 | −5.89 | −2.12 | |
| 48 | 1 | 31 | >100 | 4.3 | 9 | 65 | 0.03 | 57 | >100 | 1.8 | 4.8 | 1.9 | |
| 49 | 42 | 96 | >100 | 2 | 59 | >100 | 31 | 56 | 82 | 20.5 | 10.15 | 18.78 | |
| 50 | 64 | >100 | >100 | 64 | >100 | >100 | 65 | >100 | >100 | 25.0 | −3.95 | 1.45 | |
| 51 | 94.5 | >100 | >100 | >100 | >100 | >100 | >100 | >100 | >100 | 38.5 | 3.20 | 3.18 | |
| 52 | 0.04 | 0.5 | >100 | 0.4 | 5.8 | >100 | 1.3 | >100 | >100 | 0.6 | 2.2 | 4.1 | |
| 53 | 0.02 | 1 | >100 | 0.12 | 6.1 | >100 | 7.1 | >100 | >100 | 2.4 | 4.3 | 1.6 | |
| 54 | 0.05 | 0.8 | 91 | 3 | 9.3 | 97 | 0.2 | 62 | >100 | 1.1 | 0.6 | 8.1 | |
| 55 | 4 | 40 | >100 | 18 | >100 | >100 | 0.07 | 0.6 | 45 | 7 | 7.41 | 26 | |
| 56 | 0.3 | 0.8 | >100 | 0.5 | >100 | >100 | 0.8 | 7 | 65 | 0.5 | −6.56 | 9.88 | |
| 57 | 0.4 | 0.9 | >100 | 7 | >100 | >100 | 0.4 | 0.9 | >100 | 2.6 | −1.94 | −1 | |
| 58 | 2 | 7 | >100 | 20 | >100 | >100 | 0.04 | 0.8 | 83 | 7.3 | 0.4 | −11.54 | |
| 59 | 53 | 95 | >100 | >100 | >100 | >100 | 74 | >100 | >100 | 63.5 | 14.41 | NA | |
| 60 | 7 | 83 | >100 | 65 | >100 | >100 | >100 | >100 | >100 | 36 | 1.4 | NA | |
| 61 | >100 | >100 | >100 | >100 | >100 | >100 | 65 | >100 | >100 | >100 | NA | NA | |
| 62 | 0.1 | 8.5 | >100 | 3 | >100 | >100 | 2 | 10 | >100 | 1.7 | | | |
| 63 | 0.05 | 6 | >100 | 16 | >100 | >100 | 0.5 | 5 | >100 | 5.52 | NA | 0.17 | |
| 64 | >100 | >100 | >100 | >100 | >100 | >100 | >100 | >100 | >100 | >100 | 4.95 | 7.43 | |
| 65 | 12 | 85 | >100 | 36 | >100 | >100 | 5 | 45 | >100 | 17.67 | 11.65 | 8.84 | |
| 66 | 1 | >100 | >100 | 4.8 | >100 | >100 | 6 | >100 | >100 | 3.93 | 8.57 | 12.98 | |
| 67 | 3.5 | 9.5 | >100 | 1.8 | 67 | >100 | 3.5 | 9 | >100 | 2.93 | 12.17 | 11.64 | |
| 68 | 33 | >100 | >100 | 9 | >100 | >100 | 0.2 | 21 | >100 | 14.1 | 11.14 | 5.80 | |
| 69 | | | | | | | | | | 40.33 | 13.14 | 12.38 | |
| 70 | | | | | | | | | | 19.65 | 5.54 | 12.17 | |
| 71 | | | | | | | | | | | 9.14 | 7.67 | |
| 72 | | | | | | | | | | | 8.08 | 8.40 | |
| 73 | | | | | | | | | | >100 | 6.60 | 12.42 | |
| 74 | 60 | >100 | >100 | 42 | 74 | >100 | 0.1 | 42 | >100 | 34.0 | NA | NA | |
| 75 | 100 | >100 | >100 | >100 | >100 | >100 | 9 | 85 | >100 | >100 | NA | 6.61 | |
| 76 | 10 | 82 | >100 | 7.5 | 65 | >100 | 3 | 9 | 90 | 6.8 | 50.50 | 75.80 | |
| 77 | 53 | >100 | >100 | 60 | >100 | >100 | 6 | 65 | >100 | 39.7 | 7.7 | 14.8 | |
| 78 | 6.5 | 45 | >100 | 30 | 80 | >100 | 20 | 75 | >100 | 18.8 | 3.61 | 35.88 | |
| 79 | 30 | 74 | >100 | 30 | 65 | 95 | 0.4 | 52 | 100 | 20.1 | NA | NA | |
| 80 | 53 | >100 | >100 | >100 | >100 | >100 | 35 | 96 | >100 | >100 | NA | 5.28 | |
| 81 | | | | | | | | | | 8.50 | 7.7 | 38.2 | |
| 82 | 24 | 62 | >100 | 7 | 41 | 78 | 28 | 52 | >100 | 19.66 | 48.96 | 86.12 | |
| 83 | 30 | 60 | 90 | 10 | 56 | 84 | 9 | 66 | >100 | 16.33 | 22.38 | 63.89 | |
| 84 | 81 | >100 | >100 | >100 | >100 | >100 | 54 | >100 | >100 | >100 | 20.20 | 61.74 | |
| 85 | | | | | | | | | | 10.67 | 3.18 | 7.89 | |
| 86 | | | | | | | | | | >100 | 5.98 | −10.22 | |
| 87 | >100 | >100 | >100 | >100 | >100 | >100 | 10 | >100 | >100 | >100 | 42.24 | 87.80 | |
| 88 | >100 | >100 | >100 | >100 | >100 | >100 | 6 | >100 | >100 | >100 | NA | NA | |
| 89 | >100 | >100 | >100 | >100 | >100 | >100 | 54 | >100 | >100 | >100 | NA | 4.67 | |
| 90 | 38 | 98 | >100 | 38 | 65 | >100 | 0.06 | 48 | 98 | 25.35 | 61.63 | 98.85 | |
| 91 | 35 | >100 | >100 | 51 | >100 | >100 | 21 | 89 | >100 | 35.7 | 10.73 | 25.05 | |
| 92 | >100 | >100 | >100 | >100 | >100 | >100 | 30 | 50 | >100 | >100 | 5.02 | 1.04 | |
| 93 | 50 | >100 | >100 | 40 | 88 | >100 | 7 | 55 | >100 | 32.3 | 26.26 | 76.23 | |
| 94 | 35 | 67 | >100 | 23 | 62 | 84 | 19 | 50 | 84 | 25.66 | 52.90 | 86.29 | |
| 95 | 93 | >100 | >100 | >100 | >100 | >100 | >100 | >100 | >100 | >100 | −2.89 | 4.19 | |
| 96 | >100 | >100 | >100 | >100 | >100 | >100 | 70 | >100 | >100 | >100 | 52.41 | 86.63 | |
| 97 | 100 | >100 | >100 | >100 | >100 | >100 | >100 | >100 | >100 | >100 | 5.02 | 5.80 | |
| 98 | | | | | | | | | | >100 | 94.01 | 129.59 | |
| 99 | NA | NA | NA | NA | NA | NA | NA | NA | NA | NA | NA | NA | |
| 100 | | | | | | | | | | | 13.63 | 40.64 | |
| 101 | >100 | >100 | >100 | 14 | >100 | >100 | >100 | >100 | >100 | >100 | 8.52 | 45.50 | |
| 102 | 5 | 9.5 | 100 | 4.8 | 10 | >100 | 4.8 | 9.5 | 60 | 4.9 | 79.08 | 85.52 | 0.25 |
| 103 | 60 | >100 | >100 | 40 | >100 | >100 | 0.4 | 18 | 85 | 33.5 | NA | NA | |
| 104 | NA | NA | NA | NA | NA | NA | NA | NA | NA | NA | NA | NA | |
| 105 | | | | | | | | | | 6.73 | 11.02 | 12.74 | |
| 106 | 0.44 | 107 | >100 | >100 | >100 | >100 | 5.2 | >100 | >100 | 2.8 | 2.1 | 7.2 | |
| 107 | | | | | | | | | | 13.13 | 9.70 | 0.70 | |
| 108 | 9.5 | >100 | >100 | 100 | >100 | >100 | 5 | 15 | >100 | >100 | −8.74 | −0.86 | |
| 109 | | | | | | | | | | | 8.84 | 15.41 | |
| 110 | >100 | >100 | >100 | 80 | >100 | >100 | 10 | >100 | >100 | >100 | 17.50 | 52.34 | |

1. Novel compounds of the general formula (I), 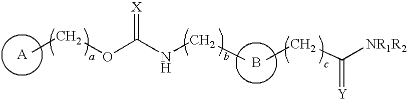
their derivatives, their analogs, their stereoisomers, their pharmaceutically acceptable salts wherein, suitable groups represented by A and B which may be substituted or unsubstituted groups are selected from aryl groups comprising phenyl, naphthyl and the like; arylalkyl groups comprising benzyl, phenylethyl, phenylpropyl and the like; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like; benzofused heteroaryl groups comprising indolyl, indolinyl, benzodioxanyl, fluorenyl, benzimidazolyl, benzotriazolyl, benzothiazolyl, quinoline, quinoxaline, acridine, phenazine, 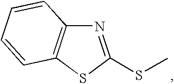
and the like. The point of attachment in case of the heteroaryl, heterocyclyl, and benzo fused heteroaryl rings to the remainder of the molecule is through one of the heteroatoms or through carbon.
Suitable groups represented by X and Y which are same or different and independently represent oxygen, sulphur or NR, wherein R represents hydrogen, hydroxy or alkyl groups comprising methyl, ethyl, n-propyl, isopropyl, n-butyl, isobutyl, t-butyl and the like.
Suitable groups represented by NR₁R₂ wherein R₁ and R₂ are same or different and independently represents hydrogen, hydroxyl, —CH₂COOEt, alkoxy groups such as methoxy, ethoxy, propoxy, n-butoxy, isobutoxy, t-butoxy and the like; benzyloxy, arylalkyl groups (such as benzyl, which are substituted by one or more groups such as —OH, and the like), acetyl, trifluoro acetyl, benzyloxy acetyl, alkyl groups comprising methyl, ethyl, n-propyl, isopropyl, n-butyl, isobutyl, t-butyl, n-pentyl and the like which are optionally substituted by one or more groups selected from alkoxy, hydroxy, substituted aryl, substituted benzyl, and —CO—NH-M, wherein M is —OH, —NO₂, —CH₂COOEt, haloalkyl, alkyl, alkenyl (such as ethenyl and the like), cycloalkyl, alkoxy and optionally substituted heteroaryl groups (for e.g., substituted with cycloalkyl); cycloalkyl groups such as cyclopropyl, cyclohexyl, cycloheptyl, cyclooctyl and the like which are optionally substituted; carboxylic acid derivatives (like esters, amides, and groups such as —O—(C═O)-M); aryl groups such as phenyl, naphthyl and the like, which are optionally substituted by the groups selected from the following —OH, —NH₂, —Ar*, —NH—CO-M, —NH—CO—Ar*, —OSO₂Me, acylamino optionally substituted by S-M, —NH—CO—NH—Ar*, wherein —Ar* is selected from the groups phenyl, heteroaryl, heterocyclyl, and benzofused hetero aryl groups which are optionally substituted with —H, —OH, —CN and —OSO₂Me; wherein M is as described earlier; heterocyclyl groups such as pyrrolidinyl, thiazolidinyl, oxazolidinyl, morpholinyl, thiomorpholinyl, piperidinyl, piperazinyl, NMethyl-piperazine and the like, which are optionally substituted by groups —(CH₂)ₑAr*, wherein Ar* is as described earlier; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like which may be substituted by groups such as —NO₂, —CH₂COOEt, cycloalkyl (such as cyclopropyl and the like), haloalkyl groups (such as trifluoro methyl and the like), —(CH₂)_{g}—CO—NH-M, wherein M is as described earlier; benzofused heteroaryl groups selected from indolyl, indolinyl, benzothiazolyl, quinoline, quinoxaline, acridine, phenazine and the like which are optionally substituted. e and f are integers in the range of 0 to 2
R₁ and R₂ are optionally fused to form a cyclic ring, which is selected from the heterocyclyl groups pyrrolidinyl, imidazolidinyl, pyrazolidinyl, piperidinyl, morpholinyl, thiomorpholinyl, piperazinyl and the like which are optionally substituted by the groups alkyl, —CO—NH-M, wherein M is as described earlier, —(CH₂)_{g}Ar*, wherein Ar* is as described earlier or heteroaryl groups pyridyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like, which are optionally substituted or benzofused heteroaryl groups indolyl, indolinyl, benzothiazolyl, quinolinyl, quinoxalinyl, quinazolinyl, pteridinyl, acridinyl, phenazinyl, phenoxazinyl, phenothiazinyl, carbazolyl and the like which may be substituted. a, b, c and g are integers in the range of 0 to 2.
Suitable groups substituted (wherein the substitution may range from 1 position to all the available positions) on A and B are selected from halogen (fluorine, chlorine, bromine, iodine), hydroxy, nitro, cyano, azido, nitroso, amino, hydrazine, formyl, alkyl, haloalkyl, haloalkoxy, cycloalkyl, aryl (optionally substituted), alkoxy, aryloxy, acyl, acyloxy, acyloxyacyl, heterocyclyl, heteroaryl (optionally substituted), monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups and carboxylic acids or its derivatives; wherein the definition of these groups remains same as defined earlier.
Furthermore when A and B are cyclic rings, they represent substituted or unsubstituted 5 to 10 membered ring systems, and also the rings may be monocyclic or bicyclic, saturated, partially saturated or aromatic, containing 1 to 4 hetero atoms selected from O, S and N and the like.

2. Novel compounds as claimed in claim 1, are selected from a group comprising of:
1. Pyridin-3-ylmethyl{4-[2-(hydroxyamino)-2-oxoethyl]-1,3-thiazol-2-yl}carbamate;
2. 2-Thienylmethyl{4-[2-(hydroxyamino)-2-oxoethyl]-1,3-thiazol-2-yl}carbamate;
3. 3-Thienylmethyl{4-[2-(hydroxyamino)-2-oxoethyl]-1,3-thiazol-2-yl}carbamate;
4. Pyridin-4-ylmethyl{4-[2-(hydroxyamino)-2-oxoethyl]-1,3-thiazol-2-4.yl}carbamate;
5. 2,3-Dihydro-1,4-benzodioxin-2-ylmethyl{4-[2-(hydroxyamino)-2-oxoethyl]1,3-thiazol-2-yl}carbamate;
6. (5-Bromo-2-thienyl)methyl{4-[(2-(hydroxyamino)-2-oxoethyl]-1,3-thiazol-2-yl}carbamate;
7. (4-Bromo-2-thienyl)methyl{4-[2-(hydroxyamino)-2-oxoethyl]-1,3-thiazol-2-yl}carbamate;
8. (4-Methyl-1,3-thiazol-5-yl)methyl{4-[2-(hydroxyamino)-2-oxoethyl]-1,3-thiazol-2-yl}carbamate;
9. 1,3-Benzothiazol-2-ylmethyl{4-[2-(hydroxyamino)-2-oxoethyl]-1,3-thiazol-2-yl}carbamate;
10. Pyridin-3-ylmethyl[4-(2-morpholin-4-yl-2-oxoethyl)-1,3-thiazol-2-yl]carbamate
11. Pyridin-3-ylmethyl{4-[2-(1,3-benzothiazol-2-ylamino)-2-oxoethyl]-1,3-thiazol-2-yl}carbamate;
12. Pyridin-3-ylmethyl[4-(2-oxo-2-piperidin-1-ylethyl)-1,3-thiazol-2-yl]carbamate;
13. Pyridin-3-ylmethyl{4-[2-(1,4′-bipiperidin-1′-yl)-2-oxoethyl]-1,3-thiazol-2-yl}carbamate;
14. Pyridin-3-ylmethyl(4-{2-[methoxy(methyl)amino]-2-oxoethyl}-1,3-thiazol-2-yl)carbamate;
15. Pyridin-3-ylmethyl{4-[2-(methylamino)-2-oxoethyl]-1,3-thiazol-2-yl}carbamate;
16. Pyridin-3-ylmethyl{4-[(2-(dimethylamino)-2-oxoethyl]-1,3-thiazol-2-yl}carbamate;
17. Pyridin-3-ylmethyl(4-{2-[(1-benzylpiperidin-4-yl)amino]-2-oxoethyl}-1,3-thiazol-2-yl)carbamate;
18. Pyridin-3-ylmethyl(4-{2-[(2-aminophenyl)amino]-2-oxoethyl}-1,3-thiazol-2-yl)carbamate;
19. Pyridin-3-ylmethyl(4-[(2-[(2-hydroxyphenyl)amino]-2-oxoethyl]-1,3-thiazol-2-yl)carbamate;
20. Pyridin-3-ylmethyl(4-{2-[(3-hydroxyphenyl)amino]-2-oxoethyl}-1,3-thiazol-2-yl)carbamate;
21. 1,3-Benzothiazol-2-ylmethyl(4-{2-[(2-aminophenyl)amino]-2-oxoethyl}-1,3-thiazol-2-yl)carbamate;
22. Pentafluorobenzyl(4-{2-[(2-aminophenyl)amino]-2-oxoethyl}-1,3-thiazol-2-yl)carbamate;
23. 1,3-Thiazol-2-ylmethyl(4-{2-[(2-aminophenyl)amino]-2-oxoethyl}-1,3-thiazol-2-yl)carbamate;
24. Pyridin-4-ylmethyl(4-{(2-[(2-aminophenyl)amino]-2-oxoethyl}-1,3-thiazol-2-yl)carbamate;
25. Pyridin-4-ylmethyl(4-{(2-[(benzyloxy)amino]-2-oxoethyl}-1,3-thiazol-2-yl)carbamate;
26. Pentafluorobenzyl(4-{2-[(5-nitro-1,3-thiazol-2-yl)amino]-2-oxoethyl}-1,3-thiazol-2-yl)carbamate;
27. 3-Thienylmethyl{4-[(hydroxyamino)carbonyl]benzyl}carbamate;
28. 2-Thienylmethyl{4-[(hydroxyamino)carbonyl]benzyl}carbamate;
29. Pentafluorobenzyl{4-[(hydroxyamino)carbonyl]benzyl}carbamate;
30. 1,3-Benzothiazol-2-ylmethyl{4-[(hydroxyamino)carbonyl]benzyl}carbamate;
31. 1,3-Thiazol-2-ylmethyl{4-[(hydroxyamino)carbonyl]benzyl}carbamate;
32. 2,3-Dihydro-1,4-benzodioxin-2-ylmethyl{4-[(hydroxyamino)carbonyl]benzyl}carbamate;
33. 1H-Benzimidazol-2-ylmethyl{4-[(hydroxyamino)carbonyl]benzyl}carbamate;
34. 1H-1,2,3-Benzotriazol-1-ylmethyl{4-[(hydroxyamino)carbonyl]benzyl}carbamate;
35. 4-(Trifluoromethyl)benzyl{4-[(hydroxyamino)carbonyl]benzyl}carbamate;
36. 3,4,5-Trimethoxybenzyl{4-[(hydroxyamino)carbonyl]benzyl}carbamate;
37. Quinolin-4-ylmethyl{4-[(hydroxyamino)carbonyl]benzyl}carbamate;
38. 2,4,6-Trifluorobenzyl{4-[(hydroxyamino)carbonyl]benzyl}carbamate;
39. 2-(1,3-Benzothiazol-2-ylthio)ethyl{4-[(hydroxyamino)carbonyl]benzyl}carbamate;
40. 2-Thienylmethyl{4-[(methylamino)carbonyl]benzyl}carbamate;
41. 1H-Benzimidazol-2-ylmethyl{4-[(methylamino)carbonyl]benzyl}carbamate;
42. 2,3-Dihydro-1,4-benzodioxin-2-ylmethyl{4-[(methylamino)carbonyl]benzyl}carbamate;
43. 2-Thienylmethyl{4-[(methoxyamino)carbonyl]benzyl}carbamate;
44. Pentafluorobenzyl(4-{[methoxyamino]carbonyl}benzyl) carbamate;
45. 2,4,6-Trifluorobenzyl(4-{[hydroxy(methyl)amino]carbonyl}benzyl)carbamate;
46. 2-Thienylmethyl(4-{[hydroxy(methyl)amino]carbonyl}benzyl)carbamate;
47. Tentafluorobenzyl(4-{[hydroxy(methyl)amino]carbonyl}benzyl)carbamate;
48. 2-Thienylmethyl(4-{[methoxy(methyl)amino]carbonyl}benzyl)carbamate;
49. 3-Thienylmethyl(4-{[(2-aminophenyl)amino]carbonyl}benzyl)carbamate;
50. 3-Thienylmethyl{4-[(4-methylpiperazin-1-yl)carbonyl]benzyl}carbamate;
51. 3-Thienylmethyl[4-(morpholin-4-ylcarbonyl)benzyl]carbamate;
52. 2-Thienylmethyl(4-{([4-(1,3-benzodioxol-5-ylmethyl)piperazin-1-yl]carbonyl}benzyl)carbamate;
53. 2-Thienylmethyl(4-{[(2,2-dimethoxyethyl)amino]carbonyl}benzyl)carbamate;
54. Ethyl{2-[(4-{[(2-thienylmethyloycarbonyl)amino]methyl}benzoyl)amino]-1,3-thiazol-4-yl}acetate;
55. 2-Thienylmethyl(4-{[(3-aminophenyl)amino]carbonyl}benzyl)carbamate;
56. Pentafluorobenzyl(4-{[(5-cyclopropyl-1,3,4-thiadiazol-2-yl)amino]carbonyl}benzyl)carbamate;
57. 1,3-Thiazol-2-ylmethyl(4-{[4-(1,3-benzodioxol-5-ylmethyl)piperazin-1-yl]carbonyl}benzyl)carbamate;
58. 1,3-Benzothiazol-2-ylmethyl[4-({[5-(trifluoromethyl)-1,3,4-thiadiazol-2-yl]amino}carbonyl)benzyl]carbamate;
59. 2-Thienylmethyl[4-({[5-(trifluoromethyl)-1,3,4-thiadiazol-2-yl]amino}carbonyl)benzyl]carbamate;
60. 2,3-Dihydro-1,4-benzodioxin-2-ylmethyl[{4-(piperidinopiperidin-1yl)carbonyl}benzyl]carbamate;
61. 2-Thienylmethyl(4-{[(2-hydroxyethyl)amino]carbonyl}benzyl)carbamate;
62. 2,4,6-Trifluorobenzyl(4-{[4-(1,3-benzodioxol-5-ylmethyl)piperazin-1-yl]carbonyl}benzyl)carbamate;
63. 2,4,6-Trifluorobenzyl(4-{[(5-cyclopropyl-1,3,4-thiadiazol-2-yl)amino]carbonyl}benzyl)carbamate;
64. 1,3-Thiazol-2-ylmethyl[4-({[5-(trifluoromethyl)-1,3,4-thiadiazol-2-yl]amino}carbonyl)benzyl]carbamate;
65. 1,3-Thiazol-2-ylmethyl(4-{[(5-cyclopropyl-1,3,4-thiadiazol-2-yl)amino]carbonyl}benzyl)carbamate;
66. 1,3-Benzothiazol-2-ylmethyl(4-{[4-(1,3-benzodioxol-5-ylmethyl)piperazin-1-yl]carbonyl}benzyl)carbamate;
67. 1,3-Benzothiazol-2-ylmethyl(4-{[(5-cyclopropyl-1,3,4-thiadiazol-2-yl)amino]carbonyl}benzyl)carbamate;
68. 1,3-thiazol-2-ylmethyl{4-[(4-pyrimidin-2-ylpiperazin-1-yl)carbonyl]benzyl}carbamate;
69. 2-thienylmethyl(4-{[(5-nitro-1,3-thiazol-2yl)amino]carbonyl}benzyl)carbamate;
70. 4-[(4-{[(2-thienylmethyloxycarbonyl)amino]methyl}benzoyl)amino]phenylmethanesulfonate;
71. 2-Thienylmethyl{4-[(4-pyridin-2-ylpiperazin-1-yl)carbonyl]benzyl}carbamate;
72. 2-Thienylmethyl{4-[(4-pyrimidin-2-ylpiperazin-1-yl)carbonyl]benzyl}carbamate;
73. 2-Thienylmethyl (4-{[(benzyloxy)amino]carbonyl}benzyl)carbamate;
74. Benzyl{4-[(hydroxyamino)carbonyl]pyridin-2-yl}carbamate;
75. Benzyl{5-[(hydroxyamino)carbonyl]-2-furyl}carbamate;
76. 8,8a-Dihydrocyclopenta[α]inden-8-ylmethyl{4-[(hydroxyamino)carbonyl]phenyl}carbamate;
77. 3-Thienylmethyl(4-[(2-[acetyl(hydroxy)amino]-2-oxoethyl]-1,3-thiazol-2-yl)carbamate;
78. (5-bromo-2-thienyl)methyl(4-{2-[acetyl(hydroxy)amino]-2-oxoethyl}-1,3-thiazol-2-yl)carbamate;
79. (4-Bromo-2-thienyl)methyl(4-{2-[acetyl(hydroxy)amino]-2-oxoethyl}-1,3-thiazol-2-yl)carbamate;
80. 2-Thienylmethyl(4-{2-[acetyl(hydroxy)amino]-2-oxoethyl}-1,3-thiazol-2-yl)carbamate;
81. 2-Thienylmethyl{4-[({4-[(trifluoroacetyl)amino]phenyl}amino)carbonyl]benzyl}carbamate;
82. 2-Thienylmethyl(4-{[(acetyloxy)amino]carbonyl}benzyl)carbamate;
83. 1,3-thiazol-2-ylmethyl(4-{[(acetyloxy)amino]carbonyl}benzyl)carbamate;
84. 2-Thienylmethyl[4-({[(methoxycarbonyl)oxy]amino}carbonyl)benzyl]carbamate;
85. 2-Thienylmethyl[4-({[(methoxycarbonyl)oxy](methyl)amino}carbonyl)benzyl]carbamate;
86. 2-Thienylmethyl[4-({(N-benzyloxy)(N-methoxycarbonyl)amino}carbonyl)benzyl]carbamate;
87. Pyridin-3-ylmethyl[4-(2-{[6-(hydroxyamino)-6-oxohexyl]amino}-2-oxoethyl)-1,3-thiazol-2-yl]carbamate;
88. Pyridin-3-ylmethyl[4-(2-{[4-(hydroxyamino)-4-oxobutyl]amino}-2-oxoethyl)-1,3-thiazol-2-yl]carbamate;
89. (4-Methyl-1,3-thiazol-5-yl)methyl[4-(2-{[4-(hydroxyamino)-4-oxobutyl]amino}-2-oxoethyl)-1,3-thiazol-2-yl]carbamate;
90. 1,3-Benzothiazol-2-ylmethyl[4-(2-{[6-(hydroxyamino)-6-oxohexyl]amino}-2-oxoethyl)-1,3-thiazol-2-yl]carbamate;
91. 1,3-Benzothiazol-2-ylmethyl[4-(2-{[4-(hydroxyamino)-4-oxobutyl]amino}-2-oxoethyl)-1,3-thiazol-2-yl]carbamate;
92. Pentafluorobenzyl[4-(2-{[4-(hydroxyamino)-4-oxobutyl]amino}-2-oxoethyl)-1,3-thiazol-2-yl]carbamate;
93. 1,3-Benzothiazol-2-ylmethyl[4-(2-{[5-(hydroxyamino)-5-oxopentyl]amino}-2-oxoethyl)-1,3-thiazol-2-yl]carbamate;
94. Pentafluorobenzyl[4-(2-{[6-(hydroxyamino)-6-oxohexyl]amino}-2-oxoethyl)-1,3-thiazol-2-yl]carbamate;
95. 1,3-Thiazol-2-ylmethyl[4-(2-{[4-(hydroxyamino)-4-oxobutyl]amino}-2-oxoethyl)-1,3-thiazol-2-yl]carbamate;
96. 1,3-Thiazol-2-ylmethyl[4-(2-{[6-(hydroxyamino)-6-oxohexyl]amino}-2-oxoethyl)-1,3-thiazol-2-yl]carbamate;
97. Pyridin-4-ylmethyl[4-(2-{[4-(hydroxyamino)-4-oxobutyl]amino}-2-oxoethyl)-1,3-thiazol-2-yl]carbamate;
98. Pyridin-4-ylmethyl[4-(2-{[6-(hydroxyamino)-6-oxohexyl]amino}-2-oxoethyl)-1,3-thiazol-2-yl]carbamate;
99. 2-Thienylmethyl[4-({(2S)-2-[(hydroxyamino)carbonyl]pyrrolidin-1-yl}carbonyl)benzyl]carbamate;
100. 2-Thienylmethyl[4-({[2-(hydroxyamino)-1-(4-hydroxybenzyl)-2-oxoethyl]amino}carbonyl)benzyl]carbamate;
101. 2-Thienylmethyl{4-[({4-[2-(hydroxyamino)-2-oxoethyl]-1,3-thiazol-2-yl}amino)carbonyl]benzyl}carbamate;
102. 2-Thienylmethyl[4-({[6-(hydroxyamino)-6-oxohexyl]amino}carbonyl)benzyl]carbamate;
103. 2-Thienylmethyl[4-({[6-(5-cyclopropyl-1,3,4-thiadiazol-2-yl)-6-oxohexyl]amino}carbonyl)benzyl]carbamate;
104. Pyridin-4-ylmethyl[4-(2-{[2-({[(4-cyanophenyl)amino]carbonyl}-amino)phenyl]amino}-2-oxoethyl)-1,3-thiazol-2-yl]carbamate;
105. 2-Thienylmethyl[4-({[3-({[(4-cyanophenyl)amino]carbonyl}amino)phenyl]amino}carbonyl)benzyl]carbamate;
106. 2-Thienylmethyl(4-[({(2-{1,3-benzodioxol-5-ylcarbonyl}amino)-phenyl}amino)carbonyl]benzyl)carbamate;
107. 2-Thienylmethyl[4-({[4-(acryloylamino)phenyl]amino}carbonyl)benzyl]carbamate;
108. 2-Thienylmethyl[4-({[3-({[(difluoromethyl)thio]acetyl}amino)phenyl]amino}carbonyl)benzyl]carbamate;
109. Ethyl{methoxy[4-({[(2-thienylmethoxy)carbonyl]amino}-methyl)benzoyl]amino}acetate and
110. 2-Thienylmethyl{4-[(E)-amino(hydroxyimino)-methyl]-phenyl}-carbamate.

3. A pharmaceutical composition, wherein the said composition comprises of a pharmaceutically effective amount of a novel compound of formula (I), 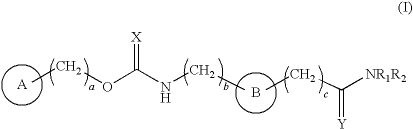
as claimed in claim 1 and a pharmaceutically acceptable carrier, diluent, excipient or solvate.

4. A pharmaceutical composition as claimed in claim 1, wherein the composition is in the form of a tablet, capsule, powder, syrup, solution, aerosol or suspension.

5. A pharmaceutical composition as claimed in claim 1, wherein the amount of the compound of claim 1 in the composition is less than 60% by weight.

6. A method inhibiting HDAC in a cell comprising said cell with an effective amount of a compound according to claim 1.

7. A method for the treatment of a condition mediated by HDAC comprising administering to a subject suffering from a condition mediated by HDAC a therapeutically effective amount of a compound according to claim 1.

8. A method for the treatment of a proliferative condition comprising administering to a subject suffering from a proliferative condition a therapeutically effective amount of a compound according to claim 1.

9. A method for the treatment of cancer comprising administering to a subject suffering from cancer a therapeutically effective amount of a compound according to claim 1.

10. A method for the treatment of psoriasis comprising administering to a subject suffering from psoriasis a therapeutically effective amount of a compound according to claim 1.
